(19) United States
(12) Patent Application Publication
(10) Pub. No.: US 2019/0223612 A1
(43) Pub. Date: Jul. 25, 2019

(54) Rockable Bed Frame

(71) Applicant: ICON Health and Fitness, Inc., Logan, UT (US)

(72) Inventors: Scott R. Wattereson, Logan, UT (US); Darren C. Ashby, Richmond, UT (US); David E. Wrobel, Farr West, UT (US); Kelly Hathaway, Hyrum, UT (US); Jared Willardson, Smithfield, UT (US); Richard Brad Ellis, Logan, UT (US); Luke Downs, Smithfield, UT (US)

(21) Appl. No.: 16/252,834

(22) Filed: Jan. 21, 2019

Related U.S. Application Data
(60) Provisional application No. 62/620,375, filed on Jan. 22, 2018.
(60) Provisional application No. 62/684,561, filed on Jun. 13, 2018.

(51) Int. Cl. A47C 21/00; A61B 5/024; A61B 5/00; A61M 21/02

(52) U.S. Cl. CPC A47C 21/006; A61B 5/024; A61B 5/6891; A61M 2205/50; A61M 2230/06; A61M 2205/103; A61M 2205/106; A61M 21/02

(57) ABSTRACT

A device for providing a kinetic input to a user includes a rockable bed frame. The rockable bed frame includes a top portion, a bottom portion, and a linear drive unit positioned between the top portion and the base portion to move the top portion and base portion relative to one another.

### CROSS-REFERENCE TO RELATED APPLICATIONS

This application claims the benefit of U.S. Provisional Patent Application No. 62/620,375, filed on Jan. 22, 2018, and this application claims the benefit of U.S. Provisional Patent Application No. 62/684,561, filed on Jun. 13, 2018, which are hereby incorporated by reference in their entireties.

### BACKGROUND

Sleep provides many benefits to humans and animals. While there is still much to learn about sleep, research suggests that during sleep restorative functions occur in the nervous, skeletal, and muscular systems. Also, memory loss has been associated with sleep deprivation suggesting that sleep plays a role in retaining memory. Some experts believe that people should get at least six hours of sleep a night. However, due to sleep disorders, some find getting adequate sleep difficult.

One type of device for determining whether a person is asleep is disclosed in U.S. Pat. No. 5,479,939 issued to Hiroyuki Ogino. In this reference, movement of a person in bed is detected without contacting the body, and time measurement is reset and started newly by a timer every time a detected movement exceeds a predetermined set value. When the measurement time of the timer exceeds a set time predetermined, it is judged that the body has fallen asleep on the bed. Meanwhile, absence or presence in bed and rough body movement are judged by detecting the fine body movement propagated by the functioning of heart and breathing of the body. Another type of system is described in U.S. Patent Publication No. 2009/0178199 issued to Andreas Brauers, et al. Each of these documents are herein incorporated by reference for all that they contain.

While these and other such systems monitor the sleep patents of a person, they do not actively aid in providing conditions to allow a person to fall asleep quicker or experience more restful sleep. The data collected by these systems may be useful in diagnosing sleep problems, however a doctor or other such medical professional may still need to provide solutions to these problems, for example via drugs or other methods.

### SUMMARY

According to some embodiments, a system for adjusting a heart rate may include a bed frame, including a base portion, a top portion disposed over the base portion and moveably connected thereto, including a yoke extending at least partially across a width of the top portion, and a drive unit fixedly attached to the base portion and to the yoke of the top portion, wherein the drive unit causes one or more of a linear movement, a rotational movement, and an elevational movement of the top portion with respect to the base portion to produce a kinetic input, an interface in communication with a heart rate monitor and the bed frame, and a processor and memory, the memory including programmed instructions to cause the processor to determine a natural heart rate of a user, determine a target heart rate for the user, and cause the bed frame to provide the kinetic input to the user to adjust the natural heart rate to the target heart rate.

The kinetic input is adjusted over a time period, and the kinetic input at a beginning of the time period is closer to the natural heart rate and the kinetic input at an end of the time period is closer to the target heart rate.

The kinetic input is changed incrementally during the time period.

The time period ends when the natural heart rate reaches the target heart rate, and the programmed instructions cause the bed frame to cease providing kinetic input while the natural heart rate is within a predetermined amount of the target heart rate.

The programmed instructions further cause the processor to determine if the natural heart rate is within a predetermined amount of the target heart rate, cause the bed frame to cease providing kinetic input when the natural heart rate is within the predetermined amount of the target heart rate, and cause the bed frame to provide the kinetic input to the user to adjust the natural heart rate to the target heart rate when the natural heart rate is outside the predetermined amount of the target heart rate.

According to some embodiments, a system for adjusting a heart rate may include a bed frame, including a base portion, including a first track fixedly attached to the base portion and extending at least partially across a width thereof, a second track fixedly attached to the base portion and extending at least partially across the width thereof, wherein the first track is oriented parallel to the second track and the first and second tracks define an axis of movement, a top portion moveably disposed over the base portion, including, at least a first roller disposed in the first track to provide for movement of the top portion with respect to the base portion along the axis of movement defined by the first and second tracks, at least a second roller disposed in the second track to provide for movement of the top portion with respect to the base portion along the axis of movement defined by the first and second tracks, a yoke extending at least partially across a width of the top portion, a motor fixedly attached to the base portion and to the yoke of the top portion, wherein the motor moves the top portion with respect to the base portion along the axis of movement defined by the first and second tracks to produce a kinetic input, a heart rate monitor, an interface in communication with the heart rate monitor and the bed frame, and a processor and memory, the memory including programmed instructions to cause the processor to determine a natural heart rate of a user, determine a target heart rate for the user, and cause the bed frame to provide the kinetic input to the user to adjust the natural heart rate to the target heart rate.

An angle of at least a head area or a foot area of the top portion of the bed frame is adjustable by the user.

The first roller includes a first wheel positioned at a first edge of the top portion and a second wheel positioned at a second edge of the top portion and the second roller includes a first wheel positioned at a first edge of the top portion and a second wheel positioned at a second edge of the top portion.

The kinetic input is adjusted over a time period and wherein the kinetic input at a beginning of the time period is closer to the natural heart rate and the kinetic input at an end of the time period is closer to the target heart rate.

The kinetic input is changed incrementally during the time period.

The time period ends when the natural heart rate reaches the target heart rate and wherein the programmed instructions cause the bed frame to cease providing kinetic input while the natural heart rate is within a predetermined amount of the target heart rate.

The programmed instructions further cause the processor to determine if the natural heart rate is within a predetermined amount of the target heart rate, cause the bed frame to cease providing kinetic input when the natural heart rate is within the predetermined amount of the target heart rate, and cause the bed frame to provide the kinetic input to the user to adjust the natural heart rate to the target heart rate when the natural heart rate is outside the predetermined amount of the target heart rate.

According to some embodiments, a system for adjusting a heart rate may include a bed frame, including a base portion, including a plurality of platforms disposed around a perimeter of the base portion, a pivot receiving member;

a top portion disposed over and rotatably connected the base portion, including a yoke extending at least partially across a width of the top portion, a pivot affixed to a midpoint of the yoke and connected to the base portion at the pivot receiving member, a plurality of rollers disposed around a perimeter of the top portion, each of the plurality of rollers in contact with one of the plurality of platforms, wherein the plurality of rollers are oriented to provide for rotational movement of the top portion with respect to the base portion about the pivot a motor fixedly attached to the base portion and to the yoke of the top portion, wherein the motor rotates the top portion with respect to the base portion about the pivot to produce a kinetic input, a hear rate monitor, an interface in communication with the heart rate monitor and the bed frame, and a processor and memory, the memory including programmed instructions to cause the processor to

determine a natural heart rate of a user, determine a target heart rate for the user, and cause the bed frame to provide the kinetic input to the user to adjust the natural heart rate to the target heart rate.

An angle of at least a head area or a foot area of the top portion of the bed frame is adjustable by the user.

The base portion includes four platforms and the top portion includes four rollers.

The base portion has a rectangular shape and each of the plurality of platforms are positioned at a corner of the base portion, and the top portion has a rectangular shape and each of the plurality of rollers is positioned at a corner of the top portion.

The target heart rate is higher than the natural heart rate.

The kinetic input is adjusted over a time period, and wherein the kinetic input at a beginning of the time period is closer to the natural heart rate and the kinetic input at an end of the time period is closer to the target heart rate.

The kinetic input is changed incrementally during the time period.

The time period ends when the natural heart rate reaches the target heart rate, and wherein the programmed instructions cause the bed frame device to cease providing kinetic input while the natural heart rate is within a predetermined amount of the target heart rate.

The programmed instructions further cause the processor to determine if the natural heart rate is within a predetermined amount of the target heart rate, cause the bed frame to cease providing kinetic input when the natural heart rate is within the predetermined amount of the target heart rate, and cause the bed frame to provide the kinetic input to the user to adjust the natural heart rate to the target heart rate when the natural heart rate is outside the predetermined amount of the target heart rate.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1 illustrates a top view of an example of a system incorporating a rockable bed frame in accordance with the present disclosure.

FIG. 2 illustrates a top view of an example of a heart rate monitor incorporated into a bed in accordance with the present disclosure.

FIGS. 3A-3E illustrate views of an example of a rockable bed frame in accordance with the present disclosure.

FIG. 4A-4E illustrate views of an example of a rockable bed frame in accordance with the present disclosure.

FIG. 5 illustrates a diagram of an example of changing a natural heart rate in accordance with the present disclosure.

FIG. 6 illustrates a block diagram of an example of an adjustment system in accordance with the present disclosure.

FIG. 7 illustrates a block diagram of an example of a method for adjusting a natural heart rate in accordance with the present disclosure.

FIG. 8 illustrates a perspective detail view of a rockable bed frame incorporating a linear drive motor in accordance with the present disclosure.

FIG. 9 illustrates a flowchart of a method of moving a bed frame in accordance with the present disclosure.

FIG. 10A-10C illustrate side views of an example of a rockable bed frame moving in accordance with the present disclosure.

FIG. 11A-11C illustrate side views of an example of a rockable bed frame moving in accordance with the present disclosure.

FIG. 12A-12E illustrate examples of rockable bed frame movement waveforms in accordance with the present disclosure.

FIG. 13 illustrates a schematic example of an anti-backlash mechanism in accordance with the present disclosure. FIG. 14 illustrates another schematic example of an anti-backlash mechanism in accordance with the present disclosure.

FIG. 15 illustrates yet another schematic example of an anti-backlash mechanism in accordance with the present disclosure.

FIG. 16 illustrates a top view of an example rockable bed frame movable by magnetic force in accordance with the present disclosure.

Throughout the drawings, identical reference numbers designate similar, but not necessarily identical, elements.

### DETAILED DESCRIPTION

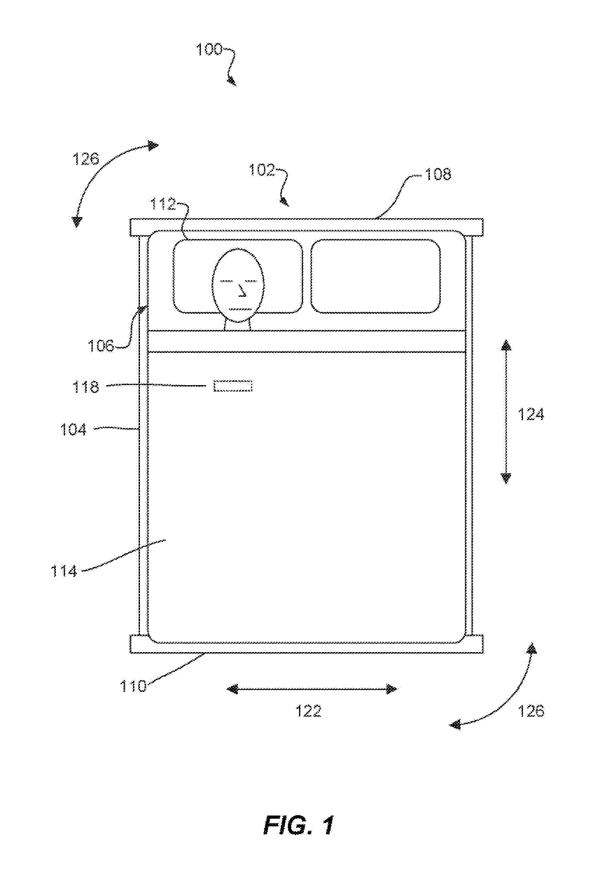

FIG. 1 depicts a top view of an example a system 100 for monitoring and controlling sleep including a bed 102. In this example, the bed 102 includes a rockable bed frame 104, bed legs, a mattress 106, a head board 108, foot board 110, bed legs (not shown) a pillow 112, and a blanket 114. When a user desires to sleep, the user may lay down on the mattress 106 and rest his or her head on the pillow 112. Depending on the temperature in the room, the user may desire to pull the blanket 114 over himself or herself. In some embodiments, one or more of the head board 108, foot board 110 and bed legs may be incorporated into or a part of the rockable bed frame 104.

Some user's may experience sleep disorders where it is difficult for a user to fall asleep or stay asleep. To assist such users, the adjustment system 100 may detect the user's heartbeat with a heart rate monitor 118. Any appropriate type of heart rate monitor may be used in accordance with the principles described in the present disclosure. For example, the user may wear a heart rate monitor that is in communication with a processor of the adjustment system 100.

The adjustment system 100 may provide kinetic input to a user, for example through the rockable bed frame 104, that mimics the target hear rate to be directed towards the user. In some examples, the processor can cause the rockable bed frame 104 to provide the kinetic input to the user. In such an example, the kinetic input can have the effect of causing the user's heart rate to slow down or speed up to the same level as the target heart rate. By bringing down the user's heart rate, the user may start to advance into the early stages of the sleeping cycle. Thus, the kinetic input may assist the user with falling asleep. In some cases, by bringing up the user's heart rate, the user may start to advance from a state of sleep or slumber to a state of wakefulness. Thus, the kinetic input may assist the user with waking form sleep.

In some embodiments, the rockable bed frame 104 may be moveable in one or more directions and/or moveable about one or more axes of movement to provide kinetic input to the user. In some examples, the bed frame may include a stationary base portion and top portion disposed over and moveable with respect to the base portion. In some embodiments, the top portion may move linearly, as indicated by arrows 122 and 124, rotationally, as indicated by arrows 126, or combinations thereof, with respect to the base portion to provide the kinetic input.

In some cases, the kinetic input may include movement away from an initiation position, while in some cases the kinetic input may include movement away from and returning to an initial position. In some cases, the kinetic input may include a reciprocal, or oscillating movement, although other forms of movement may be used as may selected by the user or as may be determined by the system 100 or a third party such as a physician. The amplitude of the movement making up the kinetic input may also be selected by the user, determined by the system 100, or determined by a third party, such as a physician.

The adjustment system 100 may automatically turn on in response to detecting a heartbeat through the heart rate monitor 118. In other examples, the adjustment system 100 automatically activates in response to detecting a heart rate when the lights are out in the room with the bed 102. In yet other examples, the adjustment system 100 automatically activates in response to detecting a heart rate during certain time periods, such as the night time or evening. In other examples, the detecting of a heart rate is not used to activate the adjustment system 100. In such cases, the time of day, identification of the user through a camera, detection of a person on the bed through weight sensors or a wireless proximity device, other mechanisms for detecting that conditions are right to activate the adjustment system 100, or combinations thereof may be used to active the adjustment system 100.

In yet other cases, the adjustment system 100 may activate in response to the user providing a command to the system to activate. For example, as the user lies down to sleep, the user may instruct the adjustment system 100 to turn on by flipping a switch, pressing a button, touching a touch screen input, sending a message through a mobile device, providing a speech command, providing instruction through another type of input mechanism, or combinations thereof.

In examples where the user wears his or her own heart rate monitor 118, the adjustment system 100 may determine the identity of the user based on an identifier of the heart rate monitor. In one example, the personal heart rate monitor 118 may include an identification code in a signal that contains the heart rate information sent to the processor. In other examples, a camera may be located in the room with the bed, and the adjustment system 100 may identify the identity of the user through the camera.

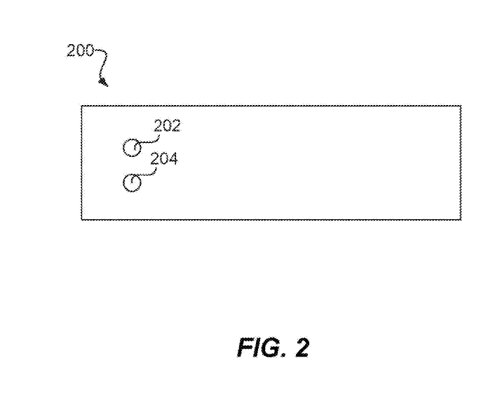

FIG. 2 illustrates a top view of an example of a heart rate monitor 200 incorporated into a bed 102 in accordance with the present disclosure. In this example, a first electrode 202 and a second electrode 204 are incorporated into a mattress 106 of the bed 102. These electrodes 202, 204 may be used to detect a voltage that represents the user's heart rate. As the user lies down, the electrodes 202, 204 may come into contact with the user's skin such that the electrodes 202, 204 can detect electrocardiography (ECG) signals of the user.

The electrodes 202, 204 may be solid metal pieces that come into contact with any appropriate parts of the user's body when the user lies down on the mattress 106. In some examples, the electrodes 202, 204 are positioned to come into contact with the user's arms, chest, legs, neck, feet, wrists, upper body, lower body, other portions of the user's body, or combinations thereof.

In other examples, the electrodes 202, 204 come into contact with the user's skin indirectly. In such examples, the electrodes 202, 204 may be buried beneath the surface of the mattress 106, but the electrodes 202, 204 come into direct contact with an electrically conductive portion of the surface of the mattress 106. Such electrically conductive portions of the mattress 106 may be flexible to provide the user with more comfort as he or she lies down on the mattress 106. In some examples, the sheets on the mattress 106 and/or the user's clothing have electrically conductive portions of fabric that come into direct contact with either the electrodes 202, 204 or electrically conductive portions of the mattress 106. Thus, while the electrodes 202, 204 may not come into direct contact with the user's skin, an electrically conductive pathway may be formed between the electrodes 202, 204 and the user's skin such that the electrodes 202, 204 can detect the user's heart rate.

In other examples, the principles described above in relation to the electrodes 202, 204 incorporated into the mattress 106 may be applied to electrodes incorporated into other portions of the bed. For example, the electrodes 202, 204 may be incorporated into the bed frame 104, the pillow 112, the blanket 114, another portion of the bed 102, or combinations thereof.

While the examples above have been described with reference to heart rate monitors that use electrical contact to determine the user's heart rate, other types of heart rate monitors may be used in accordance with the principles described herein. For example, the inductive and capacitive mechanisms for determining the user's heart rate may be used in accordance with the principles described herein. Further, the heart rate monitor or monitors need not be incorporated into the bed, and may be incorporated into, for example, an article of clothing worn by the user, a wearable device worn by the user, or some combination thereof. In some examples, a user's heart rate may be determined without contacting the user, for example, by a camera that may detect the user's heart rate through changes in the color of the user's skin and/or other methods.

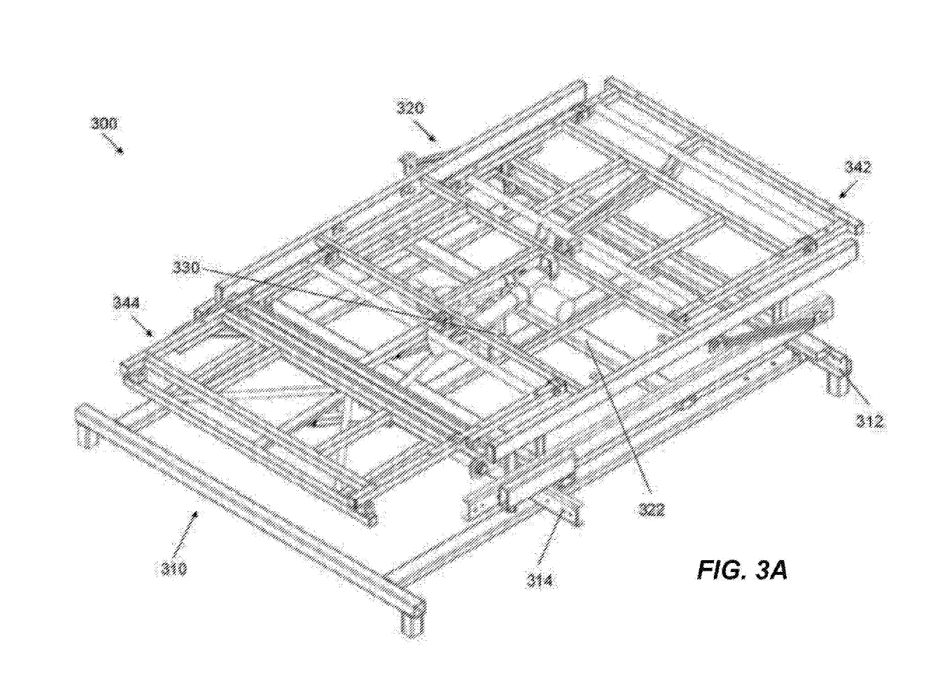

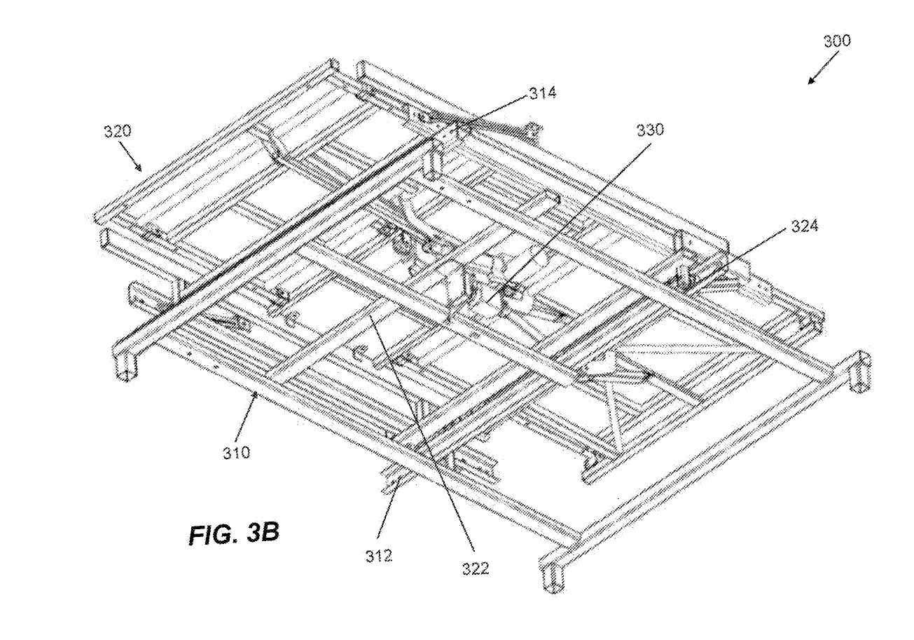

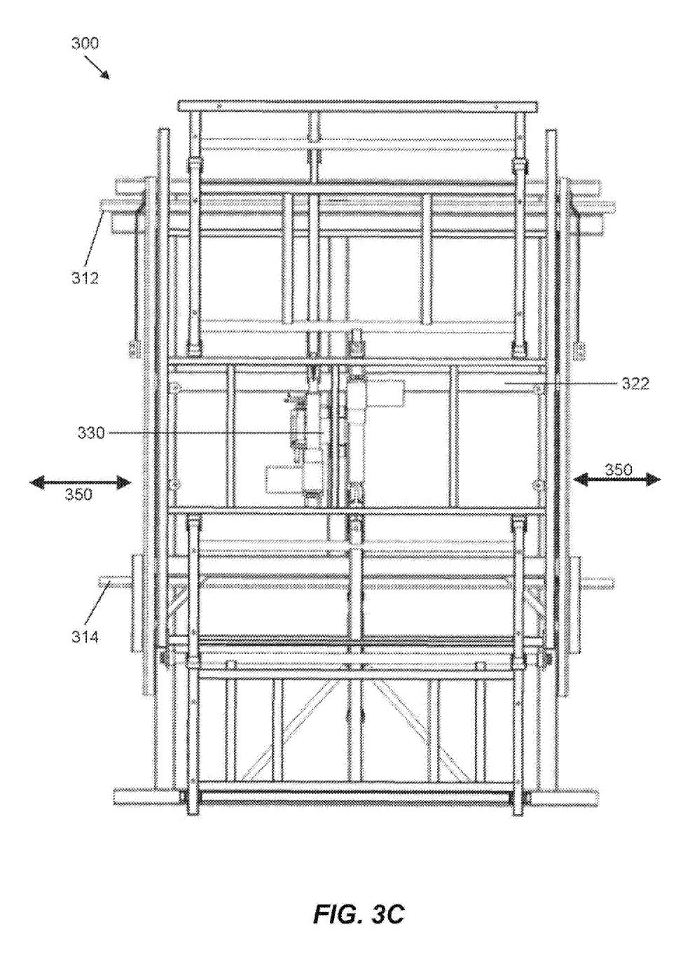

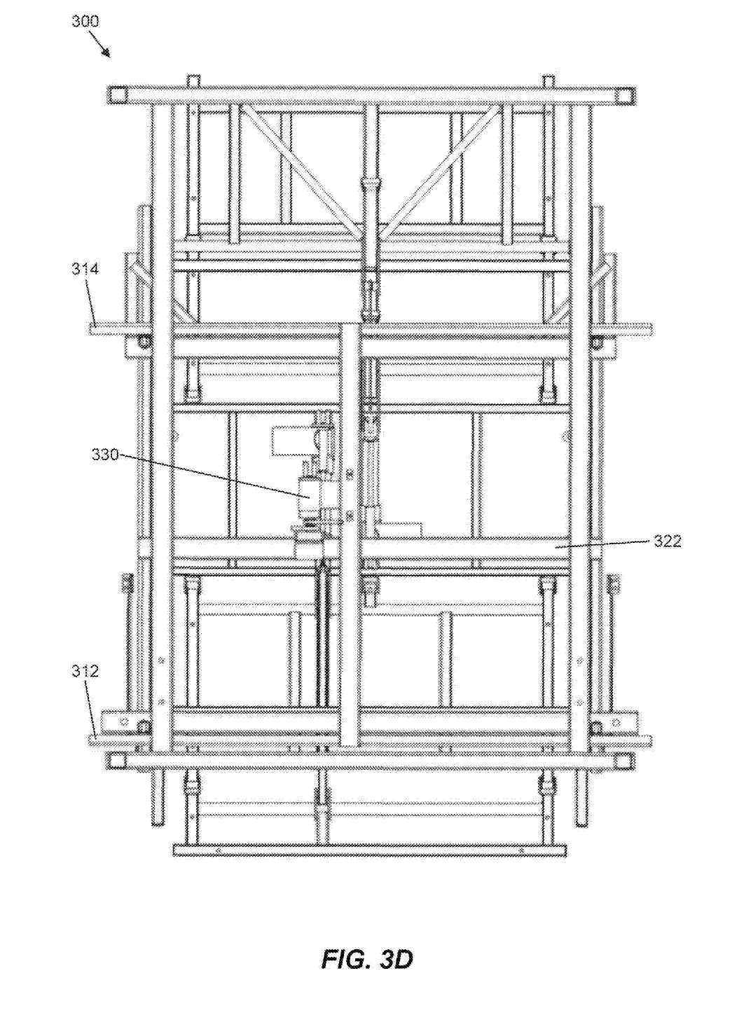

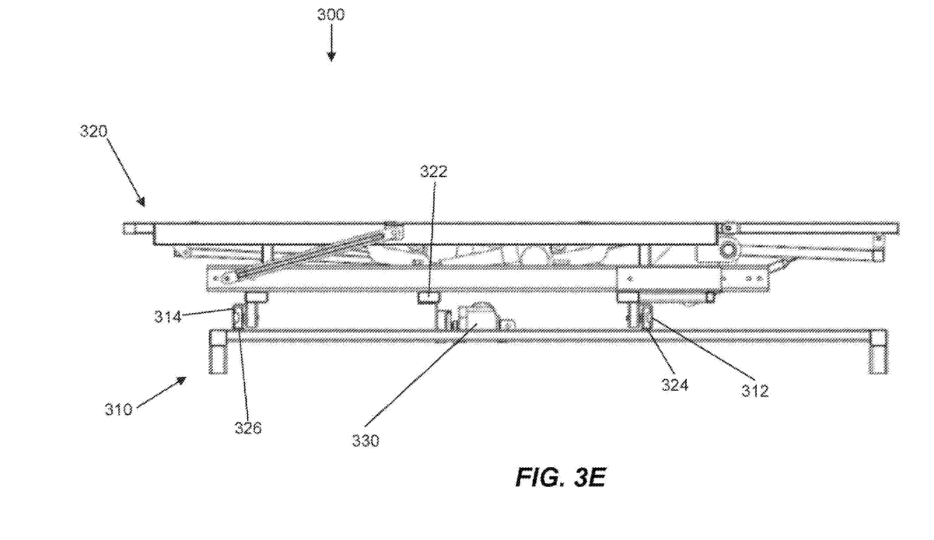

FIGS. 3A-3E illustrate a top perspective view, bottom perspective view, top view, bottom view, and side view of an exemplary rockable bed frame 300 in accordance with the present disclosure. In this example, the rockable bed frame 300 includes a stationary base portion 310 and a top portion 320 moveably disposed over the base portion 310. In this example, the bed frame 300 provides kinetic input to the user via movement of the top portion 320, which may support a mattress, with respect to the base portion 310, as described herein. The base portion includes a first track 312 and a second track 314, as can be seen in FIGS. 3A-3E, which are parallel to one another and extend laterally across a width of the base portion. Each of the tracks 312, 314 are fixedly attached to the base portion 310. In some examples, as shown in FIG. 3D, the tracks 312, 314 may extend further than the width of the base portion 310 and/or top portion 320. In the present example, and as illustrated in FIG. 3E, the tracks 312, 314 are each a metal beam having an approximately “C” shaped cross-section in order to receive and allow for movement of one or more rollers of the top portion 320 as described herein, although other forms of tracks are expressly contemplated.

The top portion 320 of the bed frame 300 may include a plurality of rollers, or wheels, which are received by and rest in the tracks 312, 314 of the base portion 310 and facilitate the movement of the top portion 320 with respect to the base portion 310. In this example, the top portion may move linearly along an axis of movement defined by the tracks 312, 314 as indicated by arrow 350 in FIG. 3A. In this example, the top portion 320 may include four wheels, positioned adjacent to the edges of the top portion and disposed in the tracks 312, 314. Two of the wheels 324, 326 are shown in FIG. 3E and may be seated in the tracks 312, 314 of the base portion 310. The side of the top portion 320 not shown may also include two wheels seated in the tracks 312, 314 in a similar configuration. In this way, the wheels support the top portion 320 on the tracks 312, 314 of the base portion 310 and moveably secure the top portion 320 to the base portion 310.

The top portion 320 also includes a yoke 322, as seen for example in FIG. 3C, that extends at least partially across the width of the top portion 320. In this example, the yoke 322 is positioned approximately centrally along the length of the top portion 320, although it may be positioned at any point along the length thereof. The bed frame 300 further includes a drive unit 330, that is fixedly attached the base portion 310, for example via a mount, and that has a drive portion fixedly attached to the yoke 322 of the top portion 320, for example as seen in FIG. 3B. In some embodiments, as in this example, the drive unit 330 may be a motor, such as an electric motor, although other drive units are expressly contemplated. When the rockable bed frame 300 is providing kinetic input to a user, the drive portion of the drive unit 330 will exert a force, as may be determined by, for example, a processor, through the yoke 322 to laterally move the top portion 320 with respect to the fixed drive unit 330 and stationary base portion 310 as indicated by arrows 350.

In some cases, the top portion 320 may further include an adjustable head 342 and/or foot portion 344. The angle of the adjustable head portion 342 and adjustable foot portion 344 may be selectively adjusted by a user, for example via a user interface.

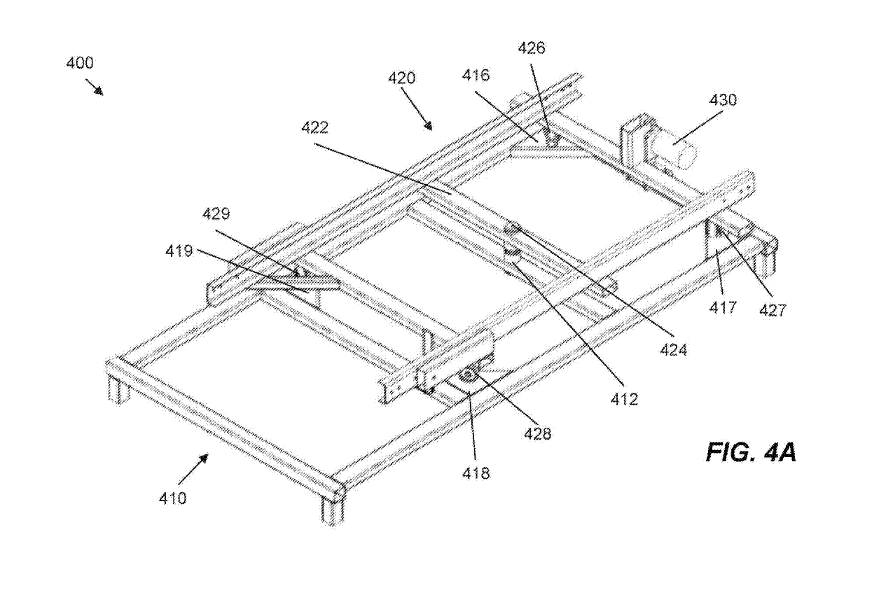

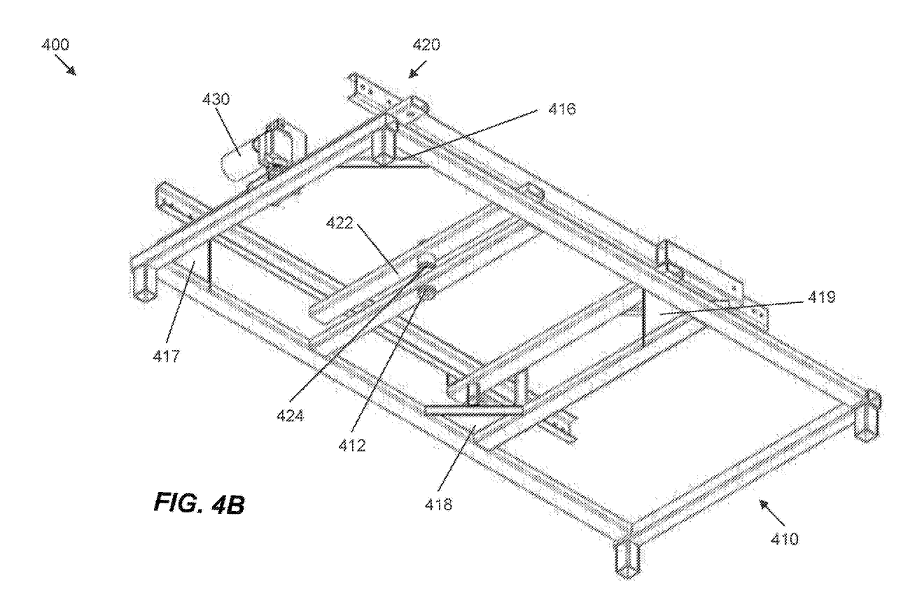

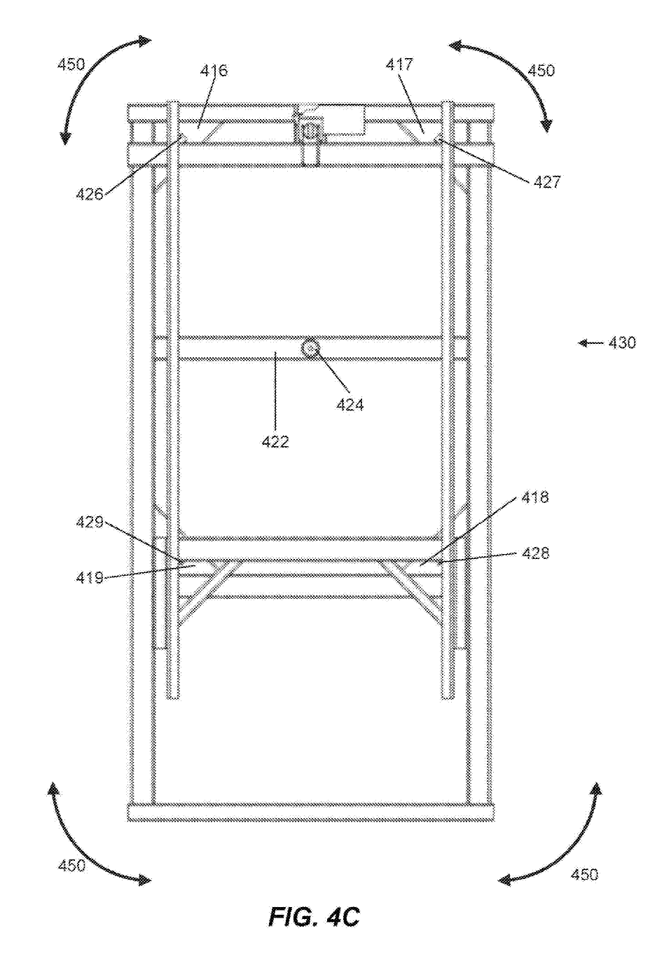

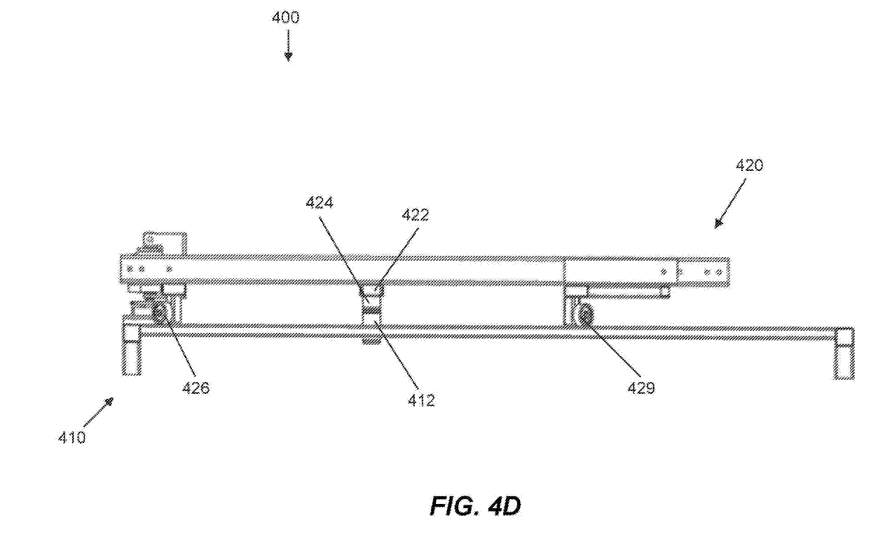

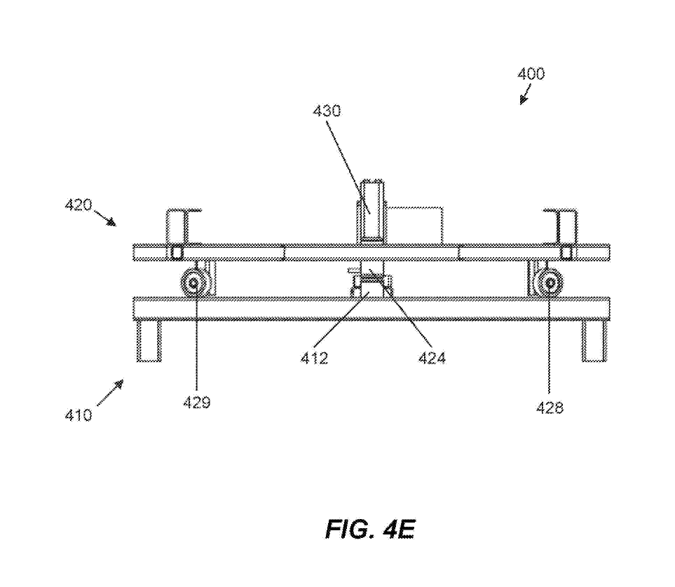

FIGS. 4A-4E illustrate a top perspective view, bottom perspective view, top view, side view, and front view of an exemplary rockable bed frame 400 in accordance with the present disclosure. In this example, the rockable bed frame 400 includes a stationary base portion 410 and a top portion 420 rotatably disposed over the base portion 410. In this example, the bed frame 400 provides kinetic input to the user via rotational movement of the top portion 420, which may support a mattress, with respect to the base portion 410, as described herein.

The base portion 410 includes a plurality of platforms disposed around a perimeter of the base portion 410. In this example, the base portion 410 includes four platforms 416, 417, 418, 419. The base portion 410 may have an approximately rectangular shape and a platform may be disposed at the corners thereof In this example, the platforms 416, 417, 418, 419 have a triangular shape and fit between the beams that make up the base portion 410, as shown in FIGS. 4A and 4C, although other platform shapes are expressly contemplated. The platforms 416, 417, 418, 419 are positioned to receive and allow for movement of one or more rollers of the top portion 420 as described herein. Although the present example includes platforms for receiving the rollers of the top portion 420, other receiving members, such as tracks are expressly contemplated. The base portion 410 further includes a pivot receiving member 412, for example as shown in FIGS. 4A-4C. The pivot receiving member 412 receives the pivot 424 of the top portion 420 as is described herein, and servers as the point about which the top portion rotates.

The top portion 420 is disposed over the base portion 410 as can be seen in FIGS. 4D-4E, and includes a plurality of rollers, or wheels, which are received by and rest and rest on the platforms 416, 417, 418, 419 of the base portion 410. In this example, there are four wheels 426, 427, 428, 429, with each wheel corresponding to and received by a platform 416, 417, 418, 419 of the base portion. The wheels 426, 427, 428, 429 and platforms 416, 417, 418, 419 facilitate the movement of the top portion 420 with respect to the base portion 410. In this example, the wheels 426, 427, 428, 429 are oriented to provide for rotational movement of the top portion 420 about the base portion 410 as indicated by the arrows 440.

The top portion 420 includes a yoke 422, as seen for example in FIG. 4A-4C, that extends at least partially across the width of the top portion 420. In this example, the yoke 422 is positioned approximately centrally along the length of the top portion 420, although it may be positioned at any point along the length thereof. The top portion 420 further includes a pivot 424, as can be seen in FIGS. 4A-4C, that is attached to the yoke 422. The pivot 424 is received by the pivot receiving member 412 of the base portion 410 and allows for rotational movement of the top portion 420 with respect to the base portion 410, as indicated by the arrows 440, about the rotational axis defined by the pivot 424

The bed frame 400 further includes a drive unit 430, that is fixedly attached the base portion 410, for example via a mount, and that has a drive portion fixedly attached to the top portion 420, for example as seen in FIG. 4A. In some embodiments, as in this example, the drive unit 430 may be a motor, such as an electric motor, although other drive units are expressly contemplated. When the rockable bed frame 400 is providing kinetic input to a user, the drive portion of the drive unit 430 will exert a force, as may be determined by, for example, a processor, to rotate the top portion 420 with respect to the fixed drive unit 430 and stationary base portion 410 about the pivot 424 as indicated by arrows 450 in FIG. 4C.

In some embodiments, the rockable bed frame 300 or 400 may also include an antenna for wireless communication with the processor of an adjustment system as described herein. In some examples, the wireless antenna may provide for communication via Bluetooth, Wi-Fi, radio waves, or any form of wireless communication now known or as may be developed in the future.

Although two specific examples of rockable bed frames are described with respect to FIGS. 3A-3E and 4A-4E, other forms of rockable bed frames that may provide kinetic input to one or more users are expressly contemplated. For example, in some embodiments a rockable bed frame may include components of both examples in order to provide kinetic input in the form of both linear and rotational movement. In some examples, a rockable bed frame may include multiple sets of tracks having different orientations and may provide linear motion in any number of directions. In some examples, a rockable bed frame may include multiple pivot points to provide rotational movement about a number of pivot points. Further, in some embodiments, the rockable bed frame may provide kinetic input in the form of raising and/or lowering the entire top portion, or parts of the top portion of the bed frame, for example via hydraulics or elevators in the base portion. In some embodiments, a rockable bed frame may include components to provide for the top portion to linearly, rotationally, elevationally, or combinations thereof with respect to the base portion. Thus, in some embodiments, the kinetic input provided by the rockable bed frame may include linear movement, rotational movement, elevational movement, or combinations thereof.

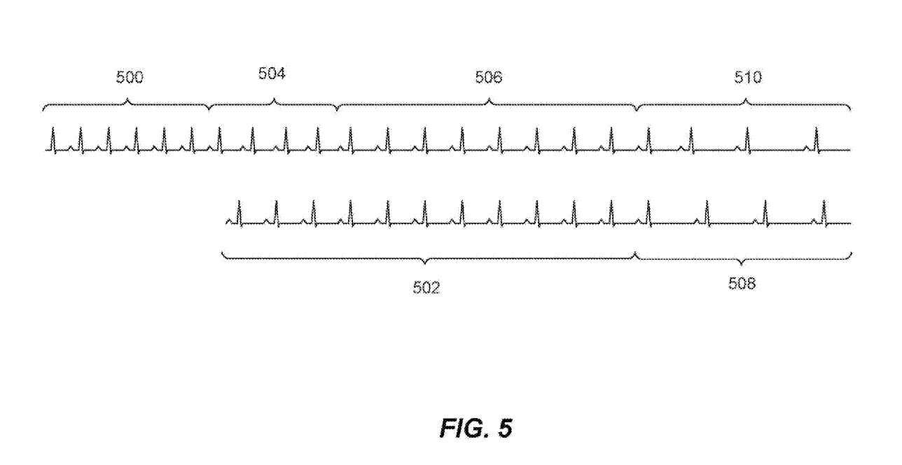

FIG. 5 illustrates a diagram of an example of changing a natural heart rate 500 in accordance with the present disclosure. In this example, the natural heart rate 500 is depicted as having a specific rate. As time passes, a first kinetic input 502 is provided to the user. The first kinetic input has a slower beat than the natural heart rate 506. As the user perceives the first kinetic input 502, the user's body causes the user's heart rate to mimic the beat of the first kinetic input 502. Thus, the user's heart rate changes during a first transition phase 504. At the end of the first transition time 504, the user's current heart rate 506 has the same rate as the first kinetic input 502. In some cases, the user's perception of the kinetic input 502 may not be a conscious perception. Thus, while the kinetic input 502 may cause a physiological response in the user, the user may not necessarily be consciously aware of the kinetic input 502 or the physiological response.

In the example of FIG. 5, the adjustment system 100 changes the user's natural heart rate to the target heart rate through multiple incremental kinetic inputs with progressively slower beats. In the illustrated example, a second kinetic input 508 is directed towards the user after the user's current heart rate 506 mimics the first kinetic input 502. The second kinetic input 508 may be closer to the target heart rate than the first kinetic input 502. As a result, the user's current heart rate 506 enters into a second transition phase 510. During the second transition phase 510, the user's current heart rate 506 slows down to mimic the beat rate of the second kinetic input 508.

This incremental process may repeat itself until the user's heart rate mimics the target heart rate with each of the kinetic inputs directed towards the user during each time increment. During each time increment, the kinetic inputs may have beat rates that progressively get closer to the target heart rate.

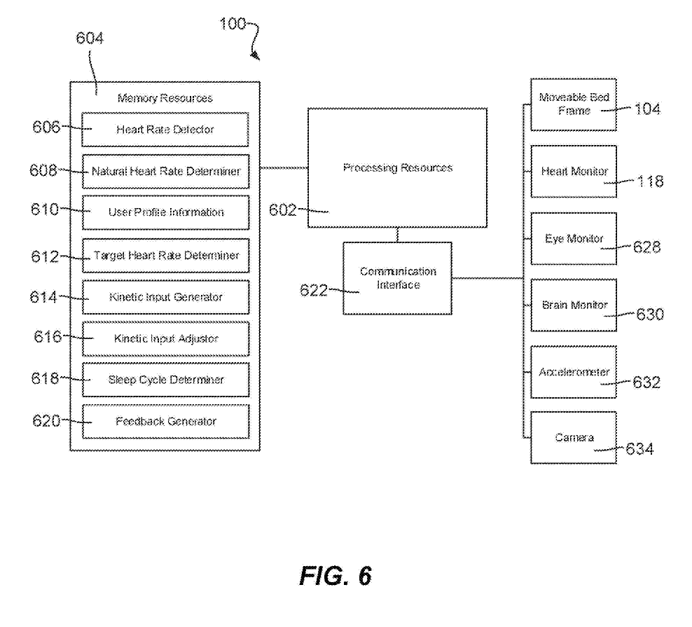

FIG. 6 illustrates a perspective view of an example of an adjustment system 100 in accordance with the present disclosure. The adjustment system 100 may include a combination of hardware and programmed instructions for executing the functions of the adjustment system 100. In this example, the adjustment system 100 includes processing resources 602 that are in communication with memory resources 604. Processing resources 602 include at least one processor and other resources used to process the programmed instructions. The memory resources 604 represent generally any memory capable of storing data such as programmed instructions or data structures used by the adjustment system 100. The programmed instructions and data structures shown stored in the memory resources 604 include a heart rate detector 606, a natural heart rate determiner 608, user profile information 610, a target heart rate determiner 612, a kinetic input generator 614, a kinetic input adjustor 616, a sleep cycle determiner 618, and a feedback generator 620.

The processing resources 602 may be in communication with communications interface 622 that communicates with external devices. Such external devices may include a rockable bed frame 104, a heart rate monitor 118, an eye monitor 628, a brain monitor 630, an accelerometer 632, a camera 634, another external device, or combinations thereof. In some examples, the processing resources 602 communicate with the external devices through a mobile device which wirelessly relays communications between the processing resources 602 and the remote devices.

The external devices may gather information or execute a task to carry out a purpose of the adjustment system 100. For example, a rockable bed frame 104 may provide kinetic input to the user in response to receiving a command from the processing resources 602. Further, the heart rate monitor 118 may collect information about the user's natural heart rate or at least the user's current heart rate, which can be used by the processing resources to determine which kinetic inputs to direct towards the user. The eye monitor 628 may be used to detect eye movement to assist the adjustment system 100 in determining whether the user is currently experiencing non-REM sleep or REM sleep.

The brain monitor 630 may be an electroencephalogram, a magnetoencephalogram, another type of brain monitor, or combinations thereof that can pick up waveforms generated by brain activity. As neurons in the brain fire, they create electrical signals that can be detected. During different stages of sleep, the brain's activity produces different types of patterns. For example, alpha waves usually have a frequency of 8.0 to 15.0 and are often exhibited during the first stage of non-REM and during REM sleep. Theta waves often exhibit a frequency of 4.0 to 7.0 hertz and are often exhibited during the second stage of non-REM sleep and REM sleep. A delta wave usually has a frequency of 1.0 to 4.0 hertz and is often exhibited during a third stage of sleep. During REM sleep, the user's brain activity often appears to be similar to when the user is awake. Thus, the brain monitor 630 may be used to determine the sleep cycle that the user is currently experiencing. As a result, the adjustment system 100 may tailor the target heart rate to be appropriate to the particular sleep stage being experienced by the user.

The accelerometer 632 may be used to determine whether the user is moving in his or her sleep. Such information may assist the adjustment system 100 in determining whether the user is in a deep sleep, REM sleep, an initial cycle of sleep, and so forth. Such information can be used to determine the appropriate target heart rate for the user based in part on the user's current sleep stage. A camera 634 may also be used to determine the user's body motions and/or restlessness and in some cases may be used to determine a user's heart rate.

Further, the communication interface may be in communication with a database that contains information about the user. An example of a database that may be compatible with the principles described herein includes the iFit program as described above. In some examples, the user information accessible through the communication interface includes the user's age, gender, body composition, height, weight, health conditions, other types of information, or combinations thereof that may be helpful in determining the appropriate target heart rate for the user.

The processing resources 602, memory resources 604 and external devices may communicate over any appropriate network and/or protocol through the communications interface 622. In some examples, the communications interface 622 includes a transceiver for wired and/or wireless communications. For example, these devices may be capable of communicating using the ZigBee protocol, Z-Wave protocol, BlueTooth protocol, Wi-Fi protocol, Global System for Mobile Communications (GSM) standard, another standard or combinations thereof. In other examples, the user can directly input some information into the adjustment system 100 through a digital input/output mechanism, a mechanical input/output mechanism, another type of mechanism or combinations thereof.

The memory resources 604 include a computer readable storage medium that contains computer readable program code to cause tasks to be executed by the processing resources 602. The computer readable storage medium may be a tangible and/or non-transitory storage medium. The computer readable storage medium may be any appropriate storage medium that is not a transmission storage medium. A non-exhaustive list of computer readable storage medium types includes non-volatile memory, volatile memory, random access memory, write only memory, flash memory, electrically erasable program read only memory, magnetic based memory, other types of memory or combinations thereof.

The heart rate detector 606 represents programmed instructions that, when executed, cause the processing resources 602 to detect the heart rate of the user. This may be accomplished in response to the heart rate monitor 118 sending information to the processing resources 602. The natural heart rate determiner 608 represents programmed instructions that, when executed, cause the processing resources 602 to determine the natural heart rate of the user. Such a determination may be based on the information from the heart rate monitor 118.

The user profile information 610 may be stored in the memory resources 604 or in a database in communication with the processing resources 602 through the communications interface 622. Such user information may include data about the user's age, gender, health conditions, weight, body compositions, and so forth that may be used to determine the target heart rate for the user.

The target heart rate determiner 612 represents programmed instructions that, when executed, cause the processing resources 602 to determine the target heart rate. In some examples, known target heart rates that can be used for a wide variety of people to assist them with sleeping or waking are used. In such an example, little personal data, if any, may be necessary to assist the user with sleeping or waking. In other examples, the target heart rate is determined based on just the natural heart rate of the user. In such examples, the target heart rate determiner 612 may use an equation to determine the target heart rate. In some cases, the equation may be a percentage of the user's resting heart rate. For example, if the user is resting on the bed 102 and the natural resting heart rate of the user is 75 beats per minute, and the equation is

0.9 (resting heart rate)=target heart rate,

than the target heart rate may be determined to be 67.5 beats per minute. While this example has been described with a specific equation, any equation, procedure, or other mechanism for determining the user's target heart rate may be used in accordance with the principles described above.

The kinetic input generator 614 represents programmed instructions that, when executed, cause the processing resources 602 to generate kinetic input that has a beat rate that is at least substantially similar to the target heart rate or at least a predetermined incremental beat customized to assist the user's heart rate to slowly adjust to the target heart rate. For example, the kinetic input generator may cause a first kinetic input to be provided to the user that is slower than the user's current heart rate, but faster than the target heart rate.

The kinetic input adjustor 616 represents programmed instructions that, when executed, cause the processing resources 602 to adjust the kinetic input as appropriate. For instance, if the kinetic input provided to the user does not represent the target heart rate, the kinetic input adjustor 616 causes the kinetic input to be adjusted such that the kinetic input progressively get closer to the target heart rate. Likewise, as the user progresses through the sleep stages and/or sleep cycles, the target heart rate may change, and the kinetic input adjustor 616 may cause kinetic inputs to change accordingly. In some cases, the kinetic input adjustor may represent programmed instructions which cause the rockable bed frame to not provide kinetic input when the natural heart rate of the user is within the predetermined amount of the target heart rate. In this case, the programmed instructions may further cause the rockable bed frame to provide the kinetic input to the user to adjust the natural heart rate to the target heart rate when the natural heart rate is not within the predetermined amount of the target heart rate.

The sleep cycle determiner 618 represents programmed instructions that, when executed, cause the processing resources 602 to determine the sleep stage and/or sleep cycle of the user. This information may be used by the target heart rate determiner 612 to determine an appropriate target heart rate.

The feedback generator 620 represents programmed instructions that, when executed, cause the processing resources 602 to generate feedback to determine the effectiveness of the target heart rate. For example, if the kinetic input generated by the adjustment system 100 causes the user to fall asleep quickly, the feedback generator 620 may determine that the generated kinetic input was effective. However, if the kinetic input causes the user to have delayed sleep, to undesirably wake up, or to take longer than desired to fall asleep, the feedback generator may adjust the target heart rate and/or the intermediary kinetic input used to help the user's heart rate arrive at the target heart rate. In some examples, the beats of the intermediary kinetic input may be adjusted. In other examples, the increment times where the intermediary kinetic input is produced may be adjusted by the feedback generator to increase the effectiveness of the adjustment system 100. Thus, the adjustment system 100 may include one or more learning algorithms for increasing the effectiveness of helping the user to sleep or wake.

Further, the memory resources 604 may be part of an installation package. In response to installing the installation package, the programmed instructions of the memory resources 604 may be downloaded from the installation package's source, such as a portable medium, a server, a remote network location, another location or combinations thereof. Portable memory media that are compatible with the principles described herein include DVDs, CDs, flash memory, portable disks, magnetic disks, optical disks, other forms of portable memory or combinations thereof. In other examples, the program instructions are already installed. Here, the memory resources 604 can include integrated memory such as a hard drive, a solid state hard drive or the like.

In some examples, the processing resources 602 and the memory resources 604 are located within the heart rate monitor 118, the rockable bed frame 104, and/or a component of the bed 102, the user's clothing, a mobile device, an external device, another type of device, or combinations thereof. The memory resources 604 may be part of any of these device's main memory, caches, registers, non-volatile memory, or elsewhere in their memory hierarchy. Alternatively, the memory resources 604 may be in communication with the processing resources 602 over a network. Further, data structures, such as libraries or databases containing user, may be accessed from a remote location over a network connection while the programmed instructions are located locally. Thus, the adjustment system 100 may be implemented with the mobile device, an external device, a phone, an electronic tablet, a wearable computing device, a head mounted device, a server, a collection of servers, a networked device, a watch, or combinations thereof. Such an implementation may occur through input/output mechanisms, such as push buttons, touch screen buttons, speech commands, dials, levers, other types of input/output mechanisms, or combinations thereof. Any appropriate type of wearable device may include, but are not limited to glasses, arm bands, leg bands, torso bands, head bands, chest straps, wrist watches, belts, earrings, nose rings, other types of rings, necklaces, garment integrated devices, other types of devices, or combinations thereof.

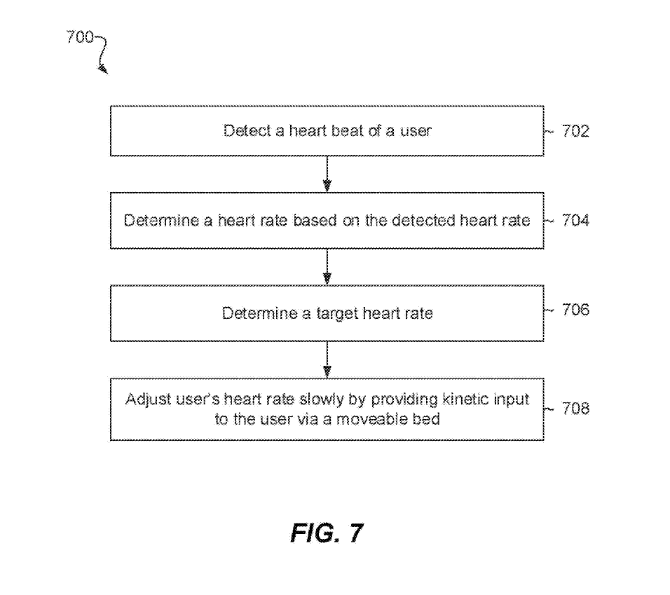

FIG. 7 illustrates a block diagram of an example of a method 700 for adjusting a natural heart rate in accordance with the present disclosure. In this example, the method 700 includes detecting 702 a heartbeat of a user, determining 704 a heart rate based on the detected heart rate, determining 706 a target heart rate, and adjusting 708 the user's heart rate slowly by directing kinetic input towards the user via the rockable bed frame.

At block 702, the heartbeat is detected. Such a heartbeat may be detected by the heart rate monitor or another type of device. At block 704, the natural heart rate is determined based at least in part on the detected heartbeat. In some examples, the heart rate is determined by counting the number of beats detected within a predetermined time period. In other examples, the signals from the heart rate monitor are filtered to remove noise or other distortions in the signal.

At block 706, the target heart rate is determined. The target heart rate may be based on applying an equation to the user's heart rate. In other examples, personal information about the user is also used to determine the target heart rate. For example, the user's age, gender, health, weight, body composition, and so forth may be used to determine the target heart rate. In some cases the target heart rate may be slower than the detected heart rate, for example when a user is attempting to sleep. However, in other cases the target heart rate may be faster than the detected heart rate, for example when a user is being woken up.

At block 708, the user's heart rate is adjusted, for example slowed or sped up, by directing kinetic input to the user via the rockable bed frame and in accordance with the principles of the present disclosure. Such kinetic input may have a beat that is at least similar to the target heart rate. In some examples, the kinetic input has a beat that is slower than the user's current heart rate, but faster than the target heart rate. In other examples, the kinetic input may have a beat that is faster than the user's current heart rate, but slower than the target heart rate. The user's heart rate may be adjusted in incremental stages or all at once.

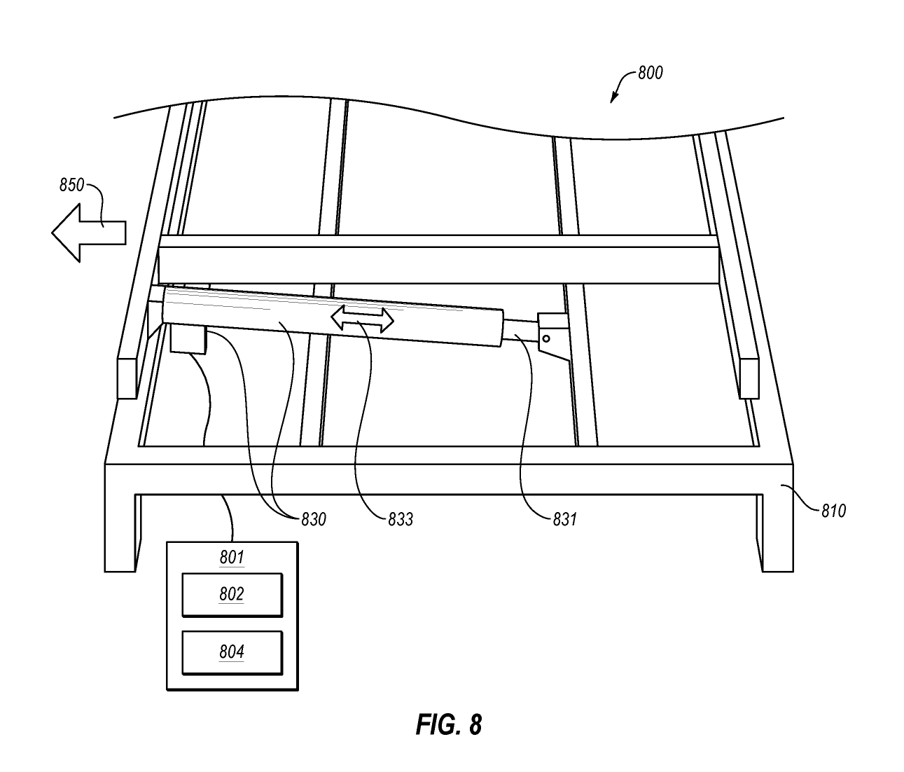

FIG. 8 is a perspective view of another embodiment of a rockable bed frame 800, according to the present disclosure. The rockable bed frame 800 includes a base portion 810 and a top portion 820, as described herein. A drive unit 830 may be positioned between at least a portion of the base portion and the top portion 820 to move the base portion 810 and top portion 820 relative to one another. In some embodiments, the drive unit 830 may be a linear actuator or linear drive unit. For example, the drive unit 830 may include worm gear that, upon rotation, moves a shaft 831 to apply an extension force or retraction force in a longitudinal direction 833 of the drive unit 830. For example, the drive unit 830 illustrated in FIG. 8 may apply an extension force to move the top portion 820 along an axis of movement indicated by arrow 850. In other examples, the drive unit 830 may apply a retraction force to move the top portion 820 along the axis of movement opposite the arrow 850.

The drive unit 830 may include a worm gear to move the shaft 831. In other examples, the drive unit 830 may be a hydraulic and/or pneumatic piston and cylinder to move the shaft 831. In yet other examples, the drive unit 830 may be a magnetic linear actuator (e.g., electromagnetic actuator) to move the shaft 831 in the longitudinal direction of the drive unit 830. In each example, a linear drive unit 830 may allow for independent control of the distance of displacement of the shaft 831, the frequency of the movement of the shaft 831, and the velocity and/or velocity curve of the shaft 831. In other words, a linear drive unit 830 may allow for independent control of the amount of displacement of the top portion 820 relative to the base portion 810, the frequency of the movement of the top portion 820 relative to the base portion 810, and the velocity of the top portion 820 relative to the base portion 810.

Conventional oscillatory beds use cams and rotary motors to provide a periodic movement of the bed with a fixed amplitude based on the size of the cam and fixed velocity curve based on the shape of the cam. A linear drive unit 830 according to the present disclosure may allow for independent control over the amplitude in each translational direction (e.g., left and right movement of the top portion 820 relative to the base portion 810) and/or rotational direction (e.g., clockwise and counterclockwise movement of the top portion 820 relative to the base portion 810). For example, the amplitude of the movement of the bed may be different in the first direction indicated by the arrow 850 from a second direction opposite that indicated by the arrow 850. In at least one example, a rockable bed frame 800 may be positioned adjacent a wall and the top portion 820 may oscillate with an amplitude only in the first direction indicated by the arrow 850. In other words, the top portion 820 may move to a first side of the base portion 810 and not pass the base portion 810 to move to an opposite side of the base portion 810.

In other embodiments, a velocity curve may be controlled independently of the amplitude. For example, the top portion 820 may move at a constant speed relative to the base portion 810 as the top portion 820 oscillates. In other examples, the top portion 820 may have a different speed in the first direction than in the second direction. In yet other examples, the top portion 820 may have a constant acceleration in the first direction and a constant velocity in the second direction. In further examples, the top portion 820 may move relative to the base portion 810 with any velocity curve achievable by the linear drive unit 830.

In yet other embodiments, a frequency of the oscillations of the top portion 820 relative to the base portion 810 may be independent of the velocity. For example, the frequency may be decreased while maintaining the velocity by extending a stroke of the shaft 831 and increasing the amplitude of the movement of the top portion 820 relative to the base portion 810. In other examples, the frequency may be increased by decreasing the amplitude or reducing the amplitude on only one side of the base portion 810.

The velocity, amplitude, and frequency of the movement of the top portion 820 relative to the base portion 810 may be independently adjusted by a computing device 801 having processing resources 802 and/or memory resources 804 in data communication with the drive unit 830. For example, the computing device 801 may be in data communication with any of the sensors or monitors describe herein, such as the heart monitor, brain monitor, eye monitor, camera, and accelerometer described in relation to FIG. 6, or other devices. In other examples, the computing device 801 may allow for user selection of an amplitude, velocity, and frequency, such as with the iFit program described herein. In yet other examples, the computing device 801 may allow for user selection of different movement recipes. An example movement recipe may include an amplitude, velocity, frequency, or combinations thereof that changes over time. A sleep assistance movement recipe may include an amplitude, velocity, and frequency that begin at a maximum value and gradually decrease over time (such as over a 30-minute duration) or that decrease in response to information received from the one or more sensors or monitors (such as information indicating a user has fallen asleep).

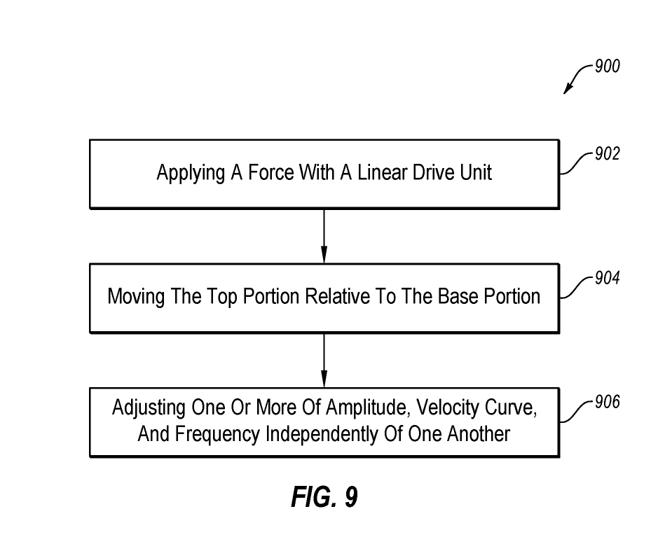

FIG. 9 illustrates a method of rocking a bed with a linear drive unit, according to the present disclosure. The method 900 includes applying a force between a top portion and a base portion of the bed with a linear drive unit at 902. The linear drive unit may include a worm gear, a piston and cylinder, a magnetic linear actuator, or other device for providing a linear force. The linear drive unit may apply an expansion force and/or a compressive force to between the top portion and the base portion of the bed to move the top portion relative to the base portion along or about any axis (i.e., translational or rotational movement) at 904. The method 900 further includes adjusting one or more of an amplitude, velocity curve, and frequency independently of one another at 906.

In some embodiments, at least one of the amplitude, velocity curve, and frequency may be adjusted independently of another of the amplitude, velocity curve, and frequency during movement of the top portion relative to the base portion. For example, the amplitude may decrease while the frequency remains constant as a user falls asleep. In other embodiments, the at least one of the amplitude, velocity curve, and frequency may be adjusted independently of another of the amplitude, velocity curve, and frequency between movements of the top portion relative to the base portion, such as selecting different values for a different user profile associated with iFit, as described herein, or when the rockable bed frame is moved to a new location and the available range of motion of the top portion of the bed is limited (e.g., by a placement near a wall).

In some embodiments, at least one of the amplitude, velocity curve, and frequency may be adjusted independently of another of the amplitude, velocity curve, and frequency by a computing device based upon information about the user received from one or more sensors or monitors in communication with the computing device. For example, at least one of the amplitude, velocity curve, and frequency may be adjusted independently of another of the amplitude, velocity curve, and frequency by a computing device based upon a heart rate of the user. In other embodiments, at least one of the amplitude, velocity curve, and frequency may be adjusted independently of another of the amplitude, velocity curve, and frequency by a computing device based upon a preselected movement recipe. For example, at least one of the amplitude, velocity curve, and frequency may be adjusted independently of another of the amplitude, velocity curve, and frequency by a computing device at preset intervals over the course of a night. In at least one example, the amplitude and velocity may decrease gradually and continuously while the frequency remains the same until the top portion ceases moving relative to the base portion. In at least one example, the amplitude may increase and decrease periodically (for example, each hour) to affect the user's heart rate at different times during each sleep cycle.

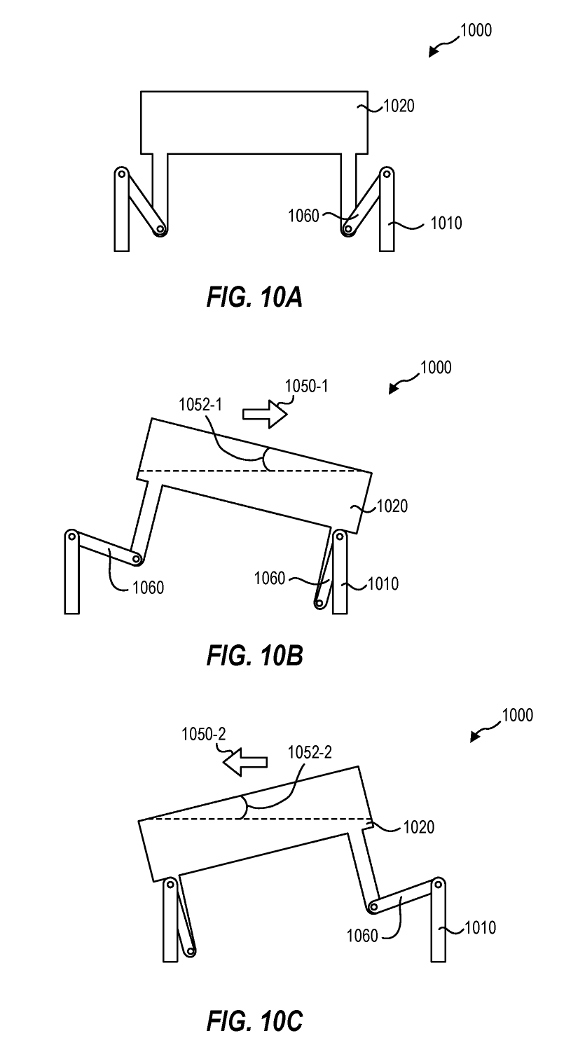

FIG. 10A through 10C illustrate another example of a rockable bed frame 1000 in accordance with the present disclosure. The rockable bed frame 1000 has a base portion 1010 and a top portion 1020, which may be similar to any of the base portions or top portions described herein. The base portion 1010 and top portion 1020 may be connected by a mechanical linkage 1060. The mechanical linkage 1060 may include at least two mechanical linkages 1060 positioned at an angle relative to one another such that when the top portion 1020 moves relative to the base portion 1010, the top portion 1020 rotates relative to the base portion 1010 as the top portion 1020 translates relative to the base portion 1010.

For example, FIG. 10B illustrates the rockable bed frame 1000 of FIG. 10A in a right position. The top portion 1020 has moved in a first direction indicated by the arrow 1050-1. The top portion 1020 may move by any drive unit, linear drive unit, or any other motive force described herein. The top portion 1020 may translate to the right relative to the base portion 1010. As the top portion 1020 translates, the mechanical linkages 1060 connecting the top portion 1020 to the base portion 1010 may rotate by different amounts, altering the relative heights of a left and right side of the top portion 1020 relative to the base portion 1010. In other words, the translation of the top portion 1020 relative to the base portion 1010 in the first direction of the arrow 1050-1 causes the mechanical linkages 1060 to tilt the top portion 1020 relative to the base portion 1010 up to a maximum of a first bed angle 1052-1 in the first direction.

In some embodiments, the first bed angle 1052-1 may be in a range having an upper value, a lower value, or upper and lower values including any of 1°, 2°, 3°, 4°, 5°, 10°, 15°, 20°, 25°, 30°, or any values therebetween. For example, the first bed angle 1052-1 may be greater than 1°. In other words, the top portion 1020 may tilt at least 1°, such that a user lying on the bed may perceive the tilting motion. In other examples, the first bed angle 1052-1 may be less than 30°. In other words, the top portion 1020 may tilt up to 30° relative to the base portion 1010, as greater than 30° may increase the risk of a user falling from the top portion 1020. In yet other examples, the first bed angle 1052-1 may be between 1° and 30°. In further examples, the first bed angle 1052-1 may be between 3° and 25°. In yet further examples, the first bed angle 1052-1 may be between 5° and 20°.

FIG. 10C illustrates the rockable bed frame 1000 of FIG. 10A with the top portion 1020 translating in a second direction indicated by the arrow 1050-2. As the top portion 1020 translates, the mechanical linkages 1060 connecting the top portion 1020 to the base portion 1010 may rotate by different amounts, altering the relative heights of a left and right side of the top portion 1020 relative to the base portion 1010. In other words, the translation of the top portion 1020 relative to the base portion 1010 in the second direction of the arrow 1050-2 causes the mechanical linkages 1060 to tilt the top portion 1020 relative to the base portion 1010 up to a maximum of a second bed angle 1052-2 in the second direction.

In some embodiments, the second bed angle 1052-2 may be in a range having an upper value, a lower value, or upper and lower values including any of 1°, 2°, 3°, 4°, 5°, 10°, 15°, 20°, 25°, 30°, or any values therebetween. For example, the second bed angle 1052-2 may be greater than 1°. In other words. the top portion 1020 may tilt at least 1°, such that a user lying on the bed may perceive the tilting motion. In other examples, the second bed angle 1052-2 may be less than 30°. In other words, the top portion 1020 may tilt up to 30° relative to the base portion 1010, as greater than 30° may increase the risk of a user falling from the top portion 1020. In yet other examples, the second bed angle 1052-2 may be between 1° and 30°. In further examples, the second bed angle 1052-2 may be between 3° and 25°. In yet further examples, the second bed angle 1052-2 may be between 5° and 20°.

In some embodiments, the first bed angle 1052-1 and second bed angle 1052-2 may be equal and in opposite directions. For example, the top portion 1020 may move between +15° and −15° relative to the base portion 1010. In other embodiments, the first bed angle 1052-1 and second bed angle 1052-2 may be different. For example, a user of the rockable bed frame 1000 may have an injury on one side of the user's body. While motion of the rockable bed frame 1000 may be therapeutic for recovery, shifting the weight of the user to the injured side of the body may be disadvantageous to recovery. In such cases, the top portion 1020 may move between a first bed angle 1052-1 of 20° and a second bed angle 1052-2 of 3°.

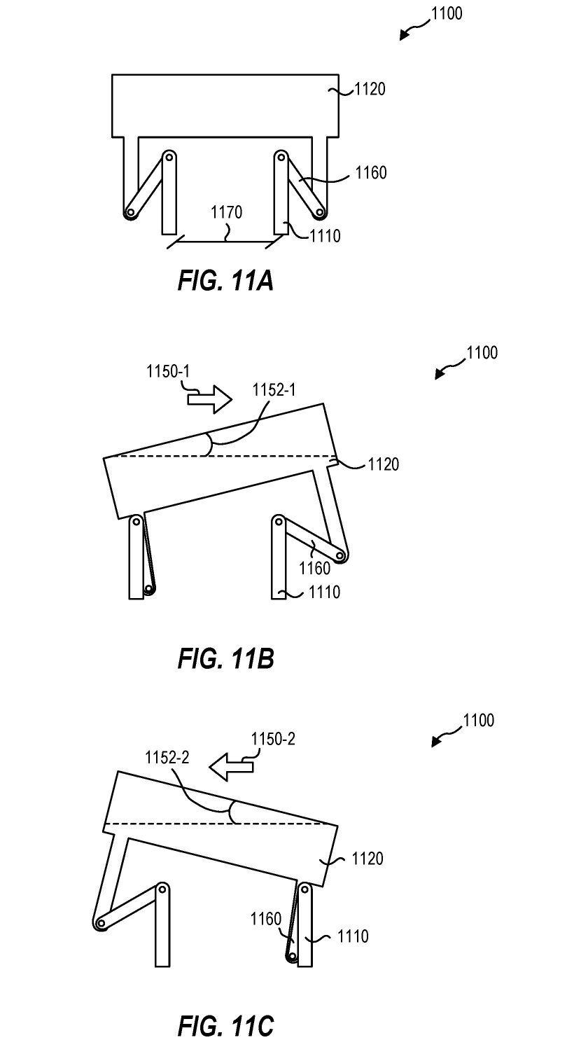

FIG. 10A through 10C illustrate an example of a rockable bed frame 1000 in which the top portion 1020 rotates outward as the top portion 1020 translates relative to the base portion 1010. In other words, the rockable bed frame 1000 moves similar to the rocking of a boat. In other examples, the rockable bed frame 1000 may move similar to the rocking of a hammock. In other words, the top portion 1020 rotates inward as the top portion 1020 translates relative to the base portion 1010. FIG. 11A through 11C illustrate such an embodiment of a rockable bed frame 1100.

FIG. 11A is a side view of a rockable bed frame 1100 with a base portion 1110 and atop portion 1120 connected by a plurality of mechanical linkages 1160 at an angle to one another. The mechanical linkages 1060 may connect the base portion 1110 to the top portion 1020 such that the base portion 1110 connection points are laterally inside the top portion 1120 connection points. In other words, a span 1170 of the base portion 1110 connection points may be less than that of the top portion 1120 connection points.

In some embodiments, the span 1170 of the base portion 1110 and/or the top portion 1120 may be adjustable, such that the orientation of the mechanical linkages 1160 may be adjustable. Altering the span 1170 of the base portion 1110 and/or the top portion 1120 relative to one another (and hence altering the orientation of the mechanical linkages 1160) may change the direction and magnitude of the bed angle during translation of the top portion 1120 relative to the base portion 1110. For example, a rockable bed frame 1100 may have a base portion 1110 and/or top portion 1120 that is adjustable such that the orientation of the mechanical linkages 1160 is adjustable.

For example, FIG. 11B illustrates the movement of the top portion 1120 of the rockable bed frame 1100 relative to the base portion 1110. The top portion 1120 may translate in the first direction indicated by the arrow 1150-1 relative to the base portion 1110. The mechanical linkages 1160 may rotate the top portion 1120 relative to the base portion 1110 during translation, such that the top portion 1120 tilts up to a first bed angle 1152-1 that is oriented inward (i.e., the top portion 1120 tilts in the direction opposite the direction of the translation). FIG. 11C illustrates the movement of the top portion 1120 of the rockable bed frame 1100 relative to the base portion 1110 as the top portion 1120 translates in the second direction indicated by the arrow 1150-2. The top portion 1120 may tilt up to a second bed angle 1152-2 that is oriented inward (i.e., the top portion 1120 tilts in the direction opposite the direction of the translation of the arrow 1150-2).

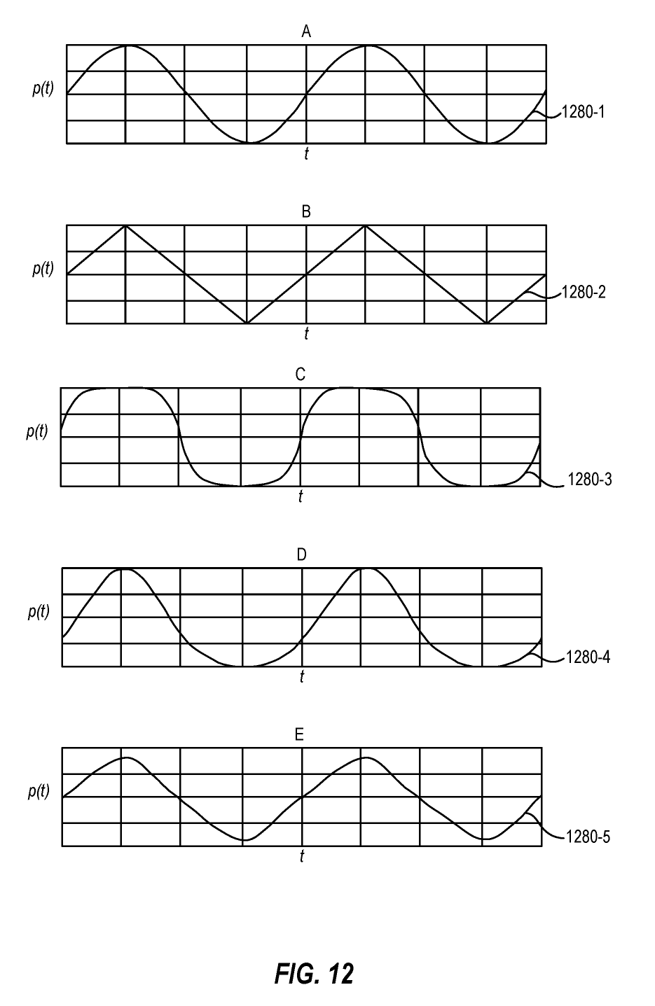

The rockable bed frames illustrated and described in relation to any embodiments herein may be driven by a linear drive motor (such as linear drive motor 830 of FIG. 8) through various waveforms. For example, FIG. 12A through 12E illustrate examples of waveforms that may describe the translational and/or rotational positions of the top portion relative to a base portion of a rockable bed frame. FIG. 12A through 12E illustrate waveforms 1280-1, 1280-2, 1280-3, 1280-4, 1280-5 according to following equations, respectively:

$$\begin{matrix}p ( t ) = D sin ( ω t ) & ( A ) \\ p ( t ) = \frac{2 D}{π} arcsin ( ω t ) & ( B ) \\ p ( t ) = \{ \begin{matrix}D ( 1 - {e}^{- β t} ) \\ D ( 1 - {e}^{β ( t - \frac{1}{2 f} )} ) \\ D ( - 1 + {e}^{- β ( t - \frac{1}{2 f} )} ) \\ D ( - 1 + {e}^{β ( t - \frac{1}{f} )} )\end{matrix} for each 1 \text{/} 4 of the period & ( C ) \\ p ( t ) = D sin ( ω t ) - l + \sqrt{{l}^{2} - ( 1 ) {cos ( ω t )}^{2}} & ( D ) \\ p ( t ) = f ( t ) & ( E )\end{matrix}$$

FIG. 12A illustrates a sine waveform 1280-1. The sine waveform 1280-1 is continuous and may provide a soothing movement for a user. FIG. 12B illustrates a discontinuous waveform 1280-2. The discontinuous waveform 1280-2 may provide the user with sudden changes in movement, stimulating circulation and aiding in recovery. In other examples, the waveform may be defined by a plurality of waveforms combined into a single combined waveform 1280-3, such as that shown in FIG. 12C. For example, the combined waveform 1280-3 is defined by different equations in four different portions of each period within the combined waveform 1280-3. In other examples, (particularly that driven by a linear drive motor), the waveform may be an asymmetrical waveform 1280-4, such as that shown in FIG. 12D. The asymmetrical waveform 1280-4 may be asymmetric in velocity, magnitude, or other property, as the rockable bed frame moves through a period. In yet other examples, the waveform 1280-5 may be a recorded waveform 1280-5 that is reconstructed by the drive motor or linear drive motor from a lookup table, such as illustrated in FIG. 12E. For example, the recorded waveform 1280-5 may be recorded by a smartphone or other electronic device with an accelerometer, gyroscope, or other device for measuring movement. Some examples of recorded waveforms 1280-5 may include using a smartphone to record the rocking of a boat on a body of water, the swinging of a hammock, the rocking of a cradle, or other periodic movement.

A recorded waveform 1280-5 may then be transmitted to the drive motor or linear drive motor of a rockable bed frame through a movable storage device (e.g., an SD card or USB storage device), a wired connection, a wireless connection, or via a remote and/or cloud storage device. For example, a recorded waveform 1280-5 may be uploaded to a user's iFit profile and subsequently downloaded to the rockable bed frame from a remote server containing the user's iFit profile.

In some embodiments, a drive motor or linear drive motor of the rockable bed frame may be driven in a first direction and, subsequently, driven in a second direction to produce the periodic and/or oscillatory motion of the rockable bed frame. The drive motor or linear drive motor may include one or more anti-backlash devices to mitigate and/or prevent backlash in the drive motor or linear drive motor that may contribute to an unintended movement of the rockable bed frame. In other examples, the drive motor or linear drive motor may be a gearless motor, such as a magnetic coupling motor, that eliminates spacing between gears within the motor, thereby reducing and/or preventing backlash. In other examples, the drive motor or linear drive motor includes an anti-backlash device that applies a force to maintain the gears of the motor in engagement with one another, such that the motor exhibits less or no backlash relative to a drive motor or linear drive motor without an anti-backlash device.

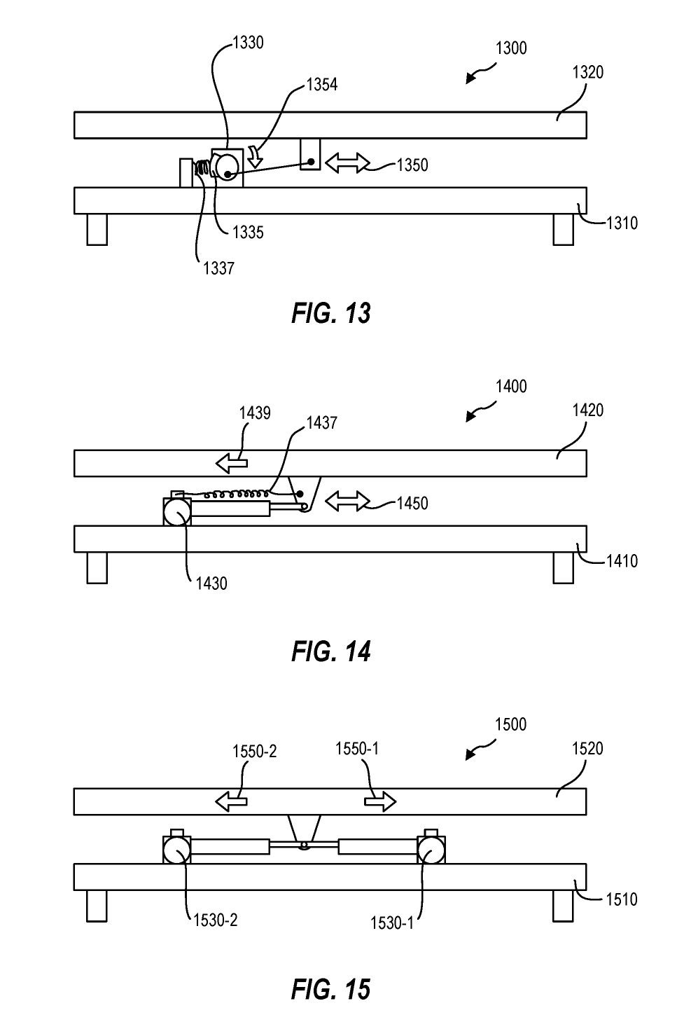

FIG. 13 is a schematic side view of a rockable bed frame 1300 with an anti-backlash device. The anti-backlash device of FIG. 13 includes a brake 1335 and a biasing element 1337 that urges the brake 1335 against the drive motor 1330. The brake 1335 may restrict the rotational movement 1354 of at least a portion of the drive motor 1330, causing the internal gears of the drive motor 1330 to remain engaged. For example, the rotational movement 1354 of the drive motor 1330 creates oscillatory translational movement of the top portion 1320 relative to the base portion 1310 as indicated by the arrow 1350. The top portion 1320 of the rockable bed frame 1300 may apply a force to the drive motor 1330 when the top portion 1320 changes translational directions at the end of the range of motion. In a conventional oscillatory system without an anti-backlash device, the force applied by the inertia of the top portion 1320 can result in a backlash and abrupt movement. Abrupt movement of the top portion 1320 may be unpleasant for a user.

In other embodiments, the inertia of the top portion relative to the drive motor and/or base portion may be controlled and/or limited by a biasing element positioned in contact with the top portion and the base portion. For example, FIG. 14 illustrates an embodiment of a rockable bed frame 1400 in accordance with the present disclosure that includes a biasing element 1437 positioned in contact with the base portion 1410 and the top portion 1420 and applying a compressive force 1439 compressing the linear drive motor 1430 between the base portion 1410 and the top portion 1420. While a linear drive motor 1430 is illustrated, a rotary drive motor or other motive force may be used.

As the top portion 1420 translates in the oscillatory movement indicated by the arrow 1450, the top portion 1420 changes direction at the ends of the range of movement. Particularly, at the far side relative to the linear drive motor 1430, any movement or tolerance in the connection of the top portion 1420 to the linear drive motor 1430 may result in a backlash that is unpleasant to a user. The biasing element 1437, such as a spring, a compressible element, a piston-and-cylinder, a magnet, or other device that applies a compressive force 1439 in response to extension, may apply a compressive force 1439 that urges the top portion 1420 toward the linear drive motor 1430, mitigating and/or preventing backlash.

FIG. 15 illustrates yet another example of a rockable bed frame 1500 with an anti-backlash device in accordance with the present disclosure. The rockable bed frame 1500 includes a base portion 1510 and a top portion 1520. The top portion 1520 translates relative to the base portion 1510 upon an application of force from at least a first drive motor 1530-1 and a second drive motor 1530-2. In some embodiments, the first drive motor 1530-1 is a rotary drive motor, and in other embodiments, the first drive motor 1530-1 is a linear drive motor. The second drive motor 1530-2 may be a rotary drive motor or the second drive motor 1530-2 may be a linear drive motor. The first drive motor 1530-1 and second drive motor 1530-2 may oppose one another, such that the first drive motor 1530-1 and second drive motor 1530-2 alternately act as the motive force on the top portion 1520 and the anti-backlash device on the opposing drive motor.

For example, the first drive motor 1530-1 may apply a contraction force to move the top portion 1520 toward the right as indicated by the first arrow 1550-1, while the second drive motor 1530-2 may retard the motion of the top portion 1520 and maintain the gears of the first drive motor 1530-1 in engagement and any movement within the connection of the first drive motor 1530-1 and the base portion 1510 and/or top portion 1520 to a minimum to reduce and/or prevent backlash. In other examples, the first drive motor 1530-1 may apply an expansion force to move the top portion 1520 toward the left as indicated by the second arrow 1550-2, while the second drive motor 1530-2 may retard the motion of the top portion 1520 and maintain the gears of the first drive motor 1530-1 in engagement and any movement within the connection of the first drive motor 1530-1 and the base portion 1510 and/or top portion 1520 to a minimum to reduce and/or prevent backlash.

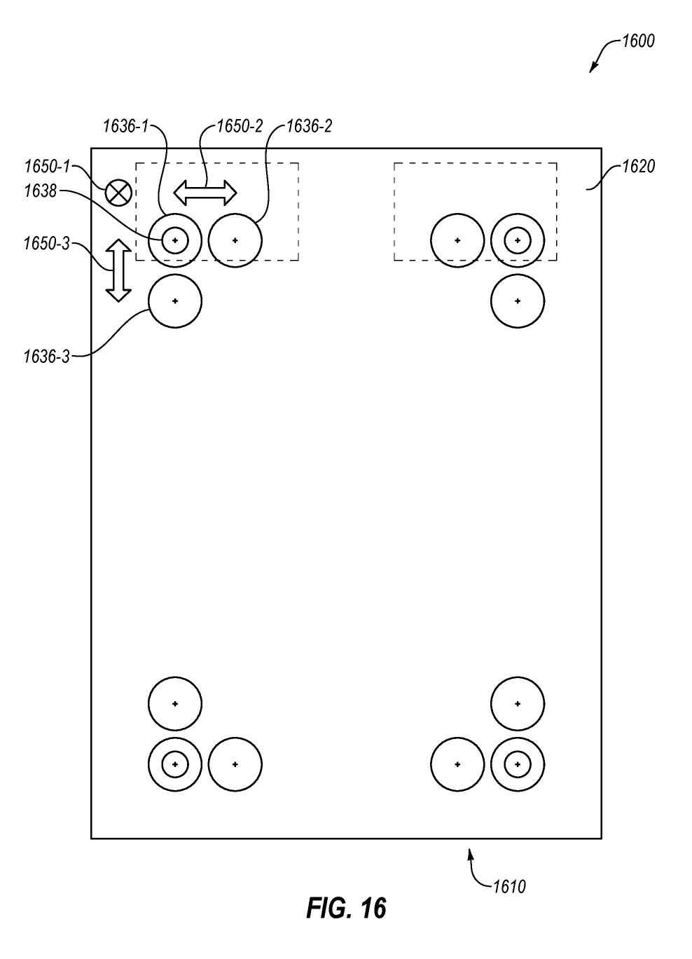

FIG. 16 is a top view schematically illustrating a rockable bed frame 1600 with a base portion 1610 and a top portion 1620 movable relative to the base portion 1610 by a plurality of electrodynamic suspension coils 1636-1, 1636-2, 1636-3. The electrodynamic suspension coils 1636-1, 1636-2, 1636-3 may be selectively activated to generate a magnetic field. The direction and magnitude of the magnetic field is controllable by altering the electric current and/or voltage applied to the electrodynamic suspension coils 1636-1, 1636-2, 1636-3. The magnetic field may generate a magnetic force on a complementary magnetic mount 1638. For example, the electrodynamic suspension coils 1636-1, 1636-2, 1636-3 and the magnetic mounts 1638 may be positioned in each corner of the rockable bed frame 1600 to support the top portion 1620 above the base portion 1610. The magnetic mounts 1638 may include or be a magnetic or ferromagnetic material, such that exposure to a magnetic field from the electrodynamic suspension coils 1636-1, 1636-2, 1636-3 may create a magnetic force between the magnetic mount 1638 and the electrodynamic suspension coils 1636-1, 1636-2, 1636-3. In some embodiments, the magnetic mounts 1638 may further include or be a flexible or resilient member, such as a spring, a compressible gas, a bushing, a tether or other object positioned between the base portion 1610 and the top portion 1620. In other embodiments, the magnetic mounts 1638 may further include rails, tracks, or other guidance elements, such as described in relation to embodiments illustrated herein, to allow movement of the top portion 1620 relative to the bottom portion 1610 in predetermined paths in response to a magnetic force.

In some embodiments, the electrodynamic suspension coils 1636-1, 1636-2, 1636-3 may include electrodynamic suspension coils 1636-1, 1636-2, 1636-3 positioned such that the selective activation of one or more of the electrodynamic suspension coils 1636-1, 1636-2, 1636-3 may apply a magnetic force in one or more axes relative to the top portion 1620 of the rockable bed frame 1600. For example, FIG. 16 illustrates a rockable bed frame 1600 with three electrodynamic suspension coils 1636-1, 1636-2, 1636-3 positioned in each corner of the top portion 1620 and a magnetic mount 1638 connected to the base portion 1610. In other examples, a rockable bed frame 1600 may have three electrodynamic suspension coils 1636-1, 1636-2, 1636-3 positioned in each corner of the base portion 1610 and a magnetic mount 1638 connected to the top portion 1620. A first electrodynamic suspension coil 1636-1 may be positioned above the magnetic mount 1638 in the z-direction. When activated, the first electrodynamic suspension coil 1636-1 may create a magnetic field that interacts with the magnetic mount 1638 to apply a z-direction magnetic force in the direction of the first arrow 1650-1. The z-direction magnetic force may be used to tilt the top portion 1620 relative to the base portion 1610.

A second electrodynamic suspension coil 1636-2 may be positioned in the x-direction relative to the first electrodynamic suspension coil 1636-1, such that the second electrodynamic suspension coil 1636-2 is positioned above the magnetic mount 1638 in the z-direction and displaced in the x-direction. When activated, the second electrodynamic suspension coil 1636-2 may create a magnetic field that interacts with the magnetic mount 1638 to apply at least a x-direction magnetic force in the direction of the second arrow 1650-2. The x-direction magnetic force may be used to translate the top portion 1620 laterally (in the side-to-side direction from the perspective of a user) relative to the base portion 1610.

A third electrodynamic suspension coil 1636-3 may be positioned in the y-direction relative to the first electrodynamic suspension coil 1636-1, such that the third electrodynamic suspension coil 1636-3 is positioned above the magnetic mount 1638 in the z-direction and displaced in the y-direction. When activated, the third electrodynamic suspension coil 1636-3 may create a magnetic field that interacts with the magnetic mount 1638 to apply at least a y-direction magnetic force in the direction of the third arrow 1650-3. The y-direction magnetic force may be used to translate the top portion 1620 longitudinally (in the head-to-toe direction from the perspective of a user) relative to the base portion 1610.

### General Description

When a healthy user lies down to sleep, the user may drift into the initial stages of sleep which are characterized by the user being in a semi-conscious state. As time progresses, the user falls into a deeper sleep. The first stages of sleep experienced by the user are referred to as non-rapid eye movement (non-REM) sleep. Often, during non-REM sleep, the user's body advances into a condition where the user's heart rate slows down, the user's breathing gets deeper and slower, and the user's muscles become more relaxed.

The final stage of sleep is rapid eye movement (REM) sleep where the user's brain activity and heart rate pick up again. During REM sleep, the user's eyes move side to side and the user may experience dreaming. REM sleep is the deepest sleep and generally, the user's muscles are often inhibited from moving during this stage of sleep. It may take a user between 90 and 120 minutes to advance through a single cycle of sleep. Upon completion of the first cycle, the user generally advances through the stages of the sleep cycle again. Often a user may complete four to six sleep cycles in a given night.

In general, the present disclosure may provide the user with system for assisting the user with sleeping and/or waking. Such a system may determine the user's natural or current heart rate with a heart rate monitor. The system may know or otherwise calculate a target heart rate to assist the user with sleeping and/or waking. Such a target heart rate may be used to assist the user with falling asleep, staying asleep, or waking from sleep. Kinetic input that adjusts the user's heart to arrive at the target heart rate may be provided to the user, for example by a rockable bed frame. Such kinetic input may have a beat that is at least similar to the target heart rate. In other examples, the kinetic input is directed at slowly causing the user's heart rate to adjust to the target heart rate by using incremental beat rates in kinetic input provided to the user for specific periods of time. The incremental beat rates may progressively adjust to the target heart rate.

The components of the adjustment system, such as the interface and heart rate monitor may be incorporated into a bed, articles of clothing, wearable devices, or combinations thereof In some examples, the heart rate monitor is independent of the bed, but is in communication with the appropriate components of the adjustment system.

An adjustment system may be well suited for individuals who have sleeping disorders, especially those types of sleeping disorders that makes it difficult for the user to relax when trying to fall asleep. However, the adjustment system as described in the present disclosure can also be used to help the user stay asleep, or to wake from sleep. For example, by continuously providing kinetic input to the user, the user's heart rate may stay at a desirable rate for sleeping. Further, as described herein, the system may be used to help the user progress through the various sleep stages and/or sleep cycles, and/or to progress to wakefulness. For example, the heart rate may be increased to help the user progress from non-REM sleep to REM sleep. Likewise, the heart rate may be adjusted to help the user move from REM sleep to non-REM sleep to help the user wake up. For example, if the adjustment system determines that the user is in REM sleep before the user's desired time period for waking, the adjustment system may provide kinetic input to the user to cause the user to transition from REM sleep to non-REM sleep. Such a system may assist the user in waking up without feeling groggy.

While the examples above have been described with reference to an adjustment system that assists a single person with sleeping, the principles described herein may be applied to assisting multiple users sleep at once. For example, the system may include multiple users where each user is associated with a dedicated heart rate monitor and/or separately moveable portion of the rockable bed frame. In some cases, a single rockable bed frame may be used to direct kinetic input to both users simultaneously. In such an example, the kinetic input may have a single beat that is customized to assist both users to fall asleep, stay asleep, and/or wake up. In other examples, a portion of a rockable bed frame or independent rockable bed frames may direct focused kinetic input to each user such that the kinetic input affects the intended user without substantially affecting the unintended user. In this way, the kinetic input directed to a user may be isolated from the other user. Such systems, may be incorporated into a double bed frame, a queen sized bed frame, a king sized bed frame, a twin sized bed frame, a fold out bed frame, another type of bed frame, or combinations thereof.

In some embodiments where multiple users are assisted, the system may include programmed instructions to cause a processor to determine the natural heart rates of a one or more users, determine separate target heart rates for each user, and cause the one or more rockable bed frames to provide individualized and independent kinetic input to each user to adjust the natural heart rate of each user to the target heart rate determined for that user.

In addition to determining the user's heart rate, the adjustment system 100 may also determine a target heart rate that may assist the user with sleeping. For example, the target heart rate, may be a heart rate that is associated with the user when the user enters into the early stages of sleeping. For example, non-REM stages of the sleep cycle are often characterized by a heart rate drop, and the target heart rate may be a heart rate exhibited by the user during such non-REM stages. In some cases, a user's target heart rate is about 6.0 to 10.0 percent lower than the user's resting heart rate. However, target heart rates may be affected by a host of factors including the user's age, weight, body composition, gender, overall fitness level, diet, other health factors, other factors, or combinations thereof.

In some embodiments, the heart rate monitor of an adjustment system may be a chest strap monitor, a wrist watch monitor, a monitor worn by the user, a monitor incorporated into the user's clothing, another type of heart rate monitor, or combinations thereof. Further, the heart rate monitor may detect electrical signals that are produced during the operation of a beating heart. Such electrical signals may be recorded by at least two electrodes in contact with the user's skin. However, other mechanisms for determining the user's heart rate may be used. For example, a microphone may be placed within a region where the microphone can pick up on the sounds made by the user's heartbeat. Further, the heart rate monitor may include a mechanism for detecting the user's pulse, and the heart rate monitor may determine the user's heart rate based on the pulse rate. While these examples have been described with reference to specific heart rate monitors, any appropriate mechanism for determining the user's heart rate may be used.

In some examples, a rockable bed frame may produce a beat that is slower than the user's natural heart rate, but faster than the target heart rate. The rockable bed frame may alternatively produce a beat that is faster than the user's natural heart rate, but slower than the target heart rate. Such intermediary heart rate kinetic inputs may be used to slowly adjust the user's heart rate to the desired target heart rate. For example, the kinetic input may mimic a beat that is 5.0 percent slower than the user's natural heart rate for a predetermined increment of time. During that increment of time, the user's heart rate may slow down to have the same rate as the beat of the kinetic input. At the conclusion of the incremental time period, another slower beat may be caused to be emitted from the rockable bed frame. As before, the user's heart rate may also slow down to mimic the rate of the subsequent kinetic input. This process may repeat itself until the user's heart rate arrives at the target heart rate, where the target heart rate continues to be mimicked, or where the kinetic input ceases.

In some cases, the kinetic input may be provided until the user's heart rate arrives at the target heart rate, whereupon the kinetic input ceases to be provided while the user's heart rate is within a predetermined amount of the target heart rate. For example, kinetic input may be provided until the user's heart rate arrives at the target heart rate and then may not be further provided unless and until the user's heart rate is no longer within, for example, 2.0 percent, 5.0 percent, or 10 percent of the target rate. At that point, kinetic input may again be provided until the user's heart rate arrives at the target heart rate, whereupon the kinetic input ceases. This process may repeat itself until the user awakens. Thus, in some examples, the heart rate monitor 118 may continue determine if the natural heart rate is within a predetermined amount of the target heart rate even when kinetic input is not being provided to the user.

The increments of time may be any appropriate length. For example, the time increments may be for 10.0 seconds, 20.0 seconds, 30.0 seconds, 1.0 minute, 2.0 minutes, another duration, or combinations thereof. Additionally, the increments of time may have different time lengths, which may depend on how much of a difference there is in the slower heart rate than the current heart rate of the user.

In some cases, the kinetic input is provided by the rockable bed frame just long enough for the user to establish a deep stage of sleep. In other cases, the kinetic input is provided through just certain stages of sleep. For example, the kinetic input may be directed to the user during just the non-REM stages of sleep or just certain stages of the non-REM sleep. Since the user's heart rate varies and often increases during REM sleep, the kinetic input may be turned off during REM sleep to avoid influencing the user's heart rate during REM sleep. In yet other cases, the kinetic input may be provided though all of the sleep cycle's stages, including during REM sleep. In other examples, the kinetic input is provided at the conclusion of the user's REM sleep to assist the user in reentering the sleep cycle, or in entering a state of wakefulness, as may be determined by, for example, a preset time.

To determine the user's heart rate, the adjustment system may have access to profile information about the user in addition to any data provided by the heart rate monitor, such as the user's weight, body composition, height, age, gender, health conditions, other factors, or combinations thereof. Such profile information may be available to the adjustment system through an iFit program available through www.ifit.com and administered through ICON Health and Fitness, Inc. located in Logan, Utah, U.S.A. An example of a program that may be compatible with the principles described in this disclosure is described in U.S. Pat. No. 7,980,996 issued to Paul Hickman. U.S. Pat. No. 7,980,996 is herein incorporated by reference for all that it discloses. However, such profile information may be available through other types of programs that contain such information. For example, such information may be gleaned from social media websites, blogs, government databases, private databases, other sources, or combinations thereof. Also, the adjustment system may record the user's heart rate through the night through the heart rate monitor and send that information to the user's profile. Such information may allow the user to determine patterns about his or her sleep, become aware of sleeping conditions, track sleeping trends, perform other tasks, or combinations thereof. Further, the recorded information may be used by the adjustment system to learn which target heart rates were the most effective for helping the user sleep. For example, if the calculated target heart rate appears to be less effective than another heart rate, the system may adapt to the other heart rate. In such examples, the target heart rates may be customized for each individual.

The adjustment system may have a single target heart rate at which the adjustment system desires to impose on the user's heart rate. In such example, the user's heart rate may be brought to that rate, and the kinetic input may cause the user's heart to maintain that rate. However, in other examples, the user's target heart rate may change over time. For example, the user's target heart rate may change depending on the stage of the user's sleep cycle. In some cases, the adjustment system may determine that the target heart rate for the user during the second stage of sleep is to be different than the target heart rate during the user's third stage of sleep. Further, the same stage in a sleep cycle may have different desirable heart rate depending on the number of sleep cycles that the user has already gone through that night. For example, during the initial sleep stages of the first cycle, the adjustment system may determine that the target heart rate is to have a first rate, while the target heart rate of the initial stages during the second sleep cycle is to have a second rate that is different than the first rate.

In some examples, the electrode may be incorporated into an article of clothing such as a garment. The garment may include at least one sensor, such as a sensor that is incorporated into the garment's fabric. In some examples, a wireless device is incorporated into the garment's fabric which can send and receive signals from other sources. Such a wireless device may be in communication with a remote device that has information about the user's sleeping habits, sleep history, personal information, other types of information, or combinations thereof that may be useful in determining where the user's heart can be to induce sleep, induce a particular stage of sleep, wake the user, or combinations thereof. The remote device may be a mobile device, a laptop, a desktop, a cloud based device, a storage device, a digital device, another type of device, or combinations thereof.

In one example, the electrode or other type of sensor may be positioned adjacent regions of the user's body through the garment to receive electrical cardio signals of the user. Such electrical cardio signals can be used to determine the user's heart rate. The cardio signals may be a user's pulse, a blood flow signal, an optical signal, an electrical timing signal used by the user's body to control the heart rate, another type of signal, or combinations thereof.

In another example, an electrode may be positioned to receive electromyography signals that detect muscle contraction. This information may be used to determine how restful the user is sleeping. In those examples where the user is tossing and turning in the night, the muscle activity can be detected, which can be used by the monitoring system to determine where the heart rate can be at to assist the user with sleeping. The sensors and/or electrodes may be positioned over the user's deltoid muscles, bicep muscles, and forearm muscles. However, the surface electromyography sensors can be positioned proximate pectoral muscles, trapezius muscles, oblique muscles, abdominal muscles, latissimus dorsi muscles, tricep muscles, hamstring muscles, quadriceps muscles, calf muscles, adductor muscles, other types of muscles, or combinations thereof. As the muscles contract, the corresponding electromyography sensor may detect an electrical signal indicating the muscle contraction.

In some cases, the clothing may also include accelerometers. Such accelerometers may be incorporated into the garment in any appropriate location to determine the types of body movements performed by the user. For example, a three-axis accelerometer may be incorporated into the garment to determine vertical and horizontal movements. The movement patterns can be analyzed to determine the user's types of movements. Accelerometers may also be used to determine a respiration count of the user. For example, at least one accelerometer positioned about the user's chest can be used to determine when the user's chest expands and contracts in accordance with the user's breathing. In other examples, a strain gauge may be incorporated into the garment, and as the user's chest expands from breathing, the strain gauge stretches. As the strain gauge stretches, it generates a signal that can be sent to the activity information device. The user's respiration data may also be used to determine how well the user is sleeping and whether a change should be made to the user's heart rate.

In some cases, a single sensor is used to determine the user's heart rate or other characteristic about the user's sleep. In other examples, multiple sensors may be incorporated into the bed, the article of clothing, a wearable device, or combinations thereof. Further, each of the sensors used to determine a characteristic about the user's sleep does not have to be of the same type of sensor. For example, a sensor may be dedicated to determining the user's pulse, another sensor for determining the respiration rate, another sensor for determining muscle contractions, another sensor to detected brain waves, another sensor detecting other types of sleep characteristics, or combination thereof.

In some cases, each sensor may be in communication with the wireless device. In some examples, each sensor is in communication with the wireless device through an independent electrically conductive medium. In other examples, the sensors communicate with each other and communicate with the wireless device through other sensors. For example, the wireless device may be in direct communication with a first sensor and indirectly in communication with a second sensor through the first sensor. The second sensor can send information towards the wireless device by sending the information to the first sensor, which then sends the information on to the wireless device. In such an example, the sensors form a network. Such a sensor network may allow sensors to communicate with each other. In some examples, such communications are bidirectional where the first sensor can send messages to the second sensor and the second sensor can send messages to the first sensor. Such networks may have any appropriate network topology, such as a daisy chain topology, a bus topology, a star topology, a mesh topology, ring topology, a tree topology, a linear topology, a fully connected topology, another type of topology, or combinations thereof.

An electrically conductive medium may include a cable or another type of wire that is disposed within channels formed within the fabric of the garment. In other examples, an electrically conductive thread is used to create an electrically conductive pathway formed in the fabric of the garment. For example, a single thread may be used to create the electrically conductive pathway. In other examples, multiple threads are used to form a patch of electrically conductive fabric capable of conducting an electrical signal. Such an electrically conductive fabric may be covered by an outer fabric layer, an inner fabric layer, a waterproof layer, a breathable layer, another type of layer, or combinations thereof. In some examples, an electrically conductive fabric is exposed in the inner or outer surfaces of the garment. In some cases, the electrodes or other types of sensors in the clothing or other type of wearable device may be held in compression against the user's body. This may be accomplished through the user of elastic material incorporated into the clothing.

While the above examples have been described with the sensors being in communication with each other or with the wireless device, any appropriate communication mechanisms may be used to enable communication between the components of the garment. For example, the sensors may be in communication with each other through fiber optic cables, wireless transceivers, other types of communication channels, or combinations thereof. In some examples, the garment includes multiple wireless devices that are capable of communication with the activity information device directly or indirectly.

Further, while the above examples have been described with reference to performing calculations and other forms of interpreting the data collected by the sensors with the activity information device, any appropriate location for performing such calculations and/or interpretations may be used in accordance with the principles described in the present disclosure. For example, such processing may occur on the mobile device, a networked device, a computing device incorporated into the garment, another type of device, or combinations thereof.

In some examples, a battery or another type of power source is incorporated into the garment and/or a wearable device. The battery may be a disposable battery or a rechargeable battery. In some cases, the garment may include an energy harvesting mechanism, such as a linear generator that can harvest the movements of the user to produce energy or a thermoelectric device that can use the thermal differential between the user's body heat and the ambient temperature of the air surrounding the user to provide energy to power the sensors of the garment. In some examples, such energy harvesting mechanisms supplement the battery or other power source in the garment or such an energy harvesting mechanism can be used to recharge such batteries.

The rockable bed frame may be moved in any movement mode described herein, including translation in any direction, rotational motion, orbital motion, or combinations thereof. In some embodiments, a top portion of the rockable bed frame may be moved relative to a lower portion of the rockable bed frame by a linear actuator or linear motor drive unit. The linear drive unit may be positioned between at least a portion of the base portion and the top portion to move the base portion and top portion relative to one another. The drive unit may include worm gear that, upon rotation, moves a shaft to apply an expansion force or contraction force in a longitudinal direction of the drive unit.

The drive unit may include a worm gear to move the shaft. In other examples, the drive unit may be a hydraulic and/or pneumatic piston and cylinder to move the shaft. In yet other examples, the drive unit may be a magnetic linear actuation (e.g., electromagnetic) to move the shaft in the longitudinal direction of the drive unit. In each example, a linear drive unit may allow for independent control of the length of the displacement of the shaft, the frequency of the movement of the shaft, and the velocity of the shaft. In other words, a linear drive unit may allow for independent control of the amount of displacement of the top portion relative to the base portion, the frequency of the movement of the top portion relative to the base portion, and the velocity of the top portion relative to the base portion.

Conventional oscillatory beds use cams and rotary motors to provide a periodic movement of the bed with a fixed amplitude based on the size of the cam and fixed velocity curve based on the shape of the cam. A linear drive unit according to the present disclosure may allow for independent control over the amplitude in each translational direction (e.g., left and right movement of the top portion relative to the base portion) and/or rotational direction (e.g., clockwise and counterclockwise movement of the top portion relative to the base portion). In at least one example, a rockable bed frame may be positioned adjacent a wall and the top portion may oscillate with an amplitude only in a first direction relative to the base portion. In other words, the top portion may move to a first side of the base portion and not pass the base portion to move to an opposite side of the base portion.

In other embodiments, a velocity curve may be controlled independently of the amplitude. For example, the top portion may move at a constant speed relative to the base portion as the top portion oscillates. In other examples, the top portion may have a different speed in the first direction than in the second direction. In yet other examples, the top portion may have a constant acceleration in the first direction and a constant velocity in the second direction. In further examples, the top portion may move relative to the base portion with any velocity curve achievable by the linear drive unit.

In yet other embodiments, a frequency of the oscillations of the top portion relative to the base portion may be independent of the velocity. For example, the frequency may be decreased while maintaining the velocity by extending a stroke of the shaft and increasing the amplitude of the movement of the top portion relative to the bottom portion. In other examples, the frequency may be increased by decreasing the amplitude or reducing the amplitude on only one side of the base portion.

The velocity, amplitude, and frequency of the movement of the top portion relative to the base portion may be independently adjusted by a computing device having processing resources and/or memory resources in data communication with the drive unit. For example, the computing device may be in data communication with any of the sensors or monitors describe herein, such as a heart monitor, brain monitor, eye monitor, camera, accelerometer, or other devices. In other examples, the computing device may allow for user selection of an amplitude, velocity, and frequency, such as with the iFit program described herein. In yet other examples, the computing device may allow for user selection of different movement recipes. An example movement recipe may include an amplitude, velocity, frequency, or combinations thereof that changes over time. A sleep assistance movement recipe may include an amplitude, velocity, and frequency that begin at a maximum value and gradually decrease over time (such as over a 30-minute duration) or that decrease in response to information received from the one or more sensors or monitors (such as information indicating a user has fallen asleep).

The rockable bed frame may include a top portion and a base portion that are connected by a mechanical linkage. The mechanical linkage may include at least two mechanical linkages positioned at an angle relative to one another such that when the top portion moves relative to the base portion, the top portion rotates relative to the base portion as the top portion translates relative to the base portion.

For example, the top portion may move by any drive unit. linear drive unit, or any other motive force described herein. The top portion may translate to the right relative to the base portion. As the top portion translates, the mechanical linkages connecting the top portion to the base portion may rotate by different amounts, altering the relative heights of a left and right side of the top portion relative to the base portion. In other words, the translation of the top portion relative to the base portion causes the mechanical linkages to tilt the top portion relative to the base portion up to a maximum of a first bed angle in the first direction.

In some embodiments, the first bed angle may be in a range having an upper value, a lower value, or upper and lower values including any of 1°, 2°, 3°, 4°, 5°, 10°, 15°, 20°, 25°, 30°, or any values therebetween. For example, the first bed angle may be greater than 1°. In other words. the top portion may tilt at least 1°, such that a user lying on the bed may perceive the tilting motion. In other examples, the first bed angle may be less than 30°. In other words, the top portion may tilt up to 30° relative to the base portion, as greater than 30° may increase the risk of a user falling from the top portion. In yet other examples, the first bed angle may be between 1° and 30°. In further examples, the first bed angle may be between 3° and 25°. In yet further examples, the first bed angle may be between 5° and 20°.

The translation of the top portion relative to the base portion in a second direction relative to the base portion causes the mechanical linkages to tilt the top portion relative to the base portion up to a maximum of a second bed angle in the second direction.

In some embodiments, the second bed angle may be in a range having an upper value, a lower value, or upper and lower values including any of 1°, 2°, 3°, 4°, 5°, 10°, 15°, 20°, 25°, 30°, or any values therebetween. For example, the second bed angle may be greater than 1°. In other words. the top portion may tilt at least 1°, such that a user lying on the bed may perceive the tilting motion. In other examples, the second bed angle may be less than 30°. In other words, the top portion may tilt up to 30° relative to the base portion, as greater than 30° may increase the risk of a user falling from the top portion. In yet other examples, the second bed angle may be between 1° and 30°. In further examples, the second bed angle may be between 3° and 25°. In yet further examples, the second bed angle may be between 5° and 20°.

In some embodiments, the first bed angle and second bed angle may be equal and in opposite directions. For example, the top portion may move between +15° and −15° relative to the base portion. In other embodiments, the first bed angle and second bed angle may be different. In such cases, the top portion may move between different bed angles on each side, such as a first bed angle of 20° and a second bed angle of 3°.

In some embodiments, a rockable bed frame may rotate outward as the top portion translates relative to the base portion. In other words, the rockable bed frame moves similar to the rocking of a boat. In other examples, the rockable bed frame may move similar to the rocking of a hammock. In other words, the top portion rotates inward as the top portion translates relative to the base portion.

The mechanical linkages may connect the base portion to the top portion such that the base portion connection points are laterally inside the top portion connection points. In other words, a span of the base portion connection points may be less than that of the top portion connection points.

In some embodiments, the span of the base portion and/or the top portion may be adjustable, such that the orientation of the mechanical linkages may be adjustable. Altering the span of the base portion and/or the top portion relative to one another (and hence altering the orientation of the mechanical linkages) may change the direction and magnitude of the bed angle during translation of the top portion relative to the base portion. For example, a rockable bed frame may have a base portion and/or top portion that is adjustable such that the orientation of the mechanical linkages is adjustable.

For example, the top portion may translate in the first direction relative to the base portion. The mechanical linkages may rotate the top portion relative to the base portion during translation, such that the top portion tilts up to a first bed angle that is oriented inward (i.e., the top portion tilts in the direction opposite the direction of the translation). When the top portion of the rockable bed frame translates relative to the base portion in the second direction, the top portion may tilt up to a second bed angle that is oriented inward (i.e., the top portion tilts in the direction opposite the direction of the translation).

The rockable bed frames illustrated and described in relation to any embodiments herein may be driven by a linear drive motor through various waveforms. Some example waveforms that may describe the translational and/or rotational positions of the top portion relative to a base portion of a rockable bed frame are shown below:

$$\begin{matrix}p ( t ) = D sin ( ω t ) & ( A ) \\ p ( t ) = \frac{2 D}{π} arcsin ( ω t ) & ( B ) \\ p ( t ) = \{ \begin{matrix}D ( 1 - {e}^{- β t} ) \\ D ( 1 - {e}^{β ( t - \frac{1}{2 f} )} ) \\ D ( - 1 + {e}^{- β ( t - \frac{1}{2 f} )} ) \\ D ( - 1 + {e}^{β ( t - \frac{1}{f} )} )\end{matrix} for each 1 \text{/} 4 of the period & ( C ) \\ p ( t ) = D sin ( ω t ) - l + \sqrt{{l}^{2} - ( 1 ) {cos ( ω t )}^{2}} & ( D ) \\ p ( t ) = f ( t ) & ( E )\end{matrix}$$

Equation (A) illustrates a sine waveform. The sine waveform is continuous and may provide a soothing movement for a user. Equation (B) illustrates a discontinuous waveform. The discontinuous waveform may provide the user with sudden changes in movement, stimulating circulation and aiding in recovery. In other examples, the waveform may be defined by a plurality of waveforms combined into a single combined waveform, such as that shown in Equation (C). For example, the combined waveform defined by different equations in four different portions of each period within the combined waveform. In other examples, (particularly that driven by a linear drive motor), the waveform may be an asymmetrical waveform, such as that shown in Equation (D). The asymmetrical waveform may be asymmetric in velocity, magnitude, or other property, as the rockable bed frame moves through a period. In yet other examples, the waveform may be a recorded waveform that is reconstructed by the drive motor or linear drive motor from a lookup table, such as illustrated in Equation (E). For example, the recorded waveform may be recorded by a smartphone or other electronic device with an accelerometer, gyroscope, or other device for measuring movement. Some examples of recorded waveforms may include using a smartphone to record the rocking of a boat on a body of water, the swinging of a hammock, the rocking of a cradle, or other periodic movement.

A recorded waveform may then be transmitted to the drive motor or linear drive motor of a rockable bed frame through a movable storage device (e.g., a Secure Digital (SD) card or Universal Serial Bus (USB) storage device), a wired connection, a wireless connection, or via a remote and/or cloud storage device. For example, a recorded waveform may be uploaded to a user's iFit profile and subsequently downloaded to the rockable bed frame from a remote server containing the user's iFit profile.

In some embodiments, a drive motor or linear drive motor of the rockable bed frame may be driven in a first direction and, subsequently, driven in a second direction to produce the periodic and/or oscillatory motion of the rockable bed frame. The drive motor or linear drive motor may include one or more anti-backlash devices to mitigate and/or prevent backlash in the drive motor or linear drive motor that may contribute to an unintended movement of the rockable bed frame. In other examples, the drive motor or linear drive motor may be a gearless motor, such as a magnetic coupling motor, that eliminates spacing between gears within the motor, thereby reducing and/or preventing backlash. In other examples, the drive motor or linear drive motor includes an anti-backlash device that applies a force to maintain the gears of the motor in engagement with one another, such that the motor exhibits less or no backlash relative to a drive motor or linear drive motor without an anti-backlash device.

In some embodiments, the anti-backlash device includes a brake and a biasing element that urges the brake against the drive motor. The brake may restrict the rotational movement of at least a portion of the drive motor, causing the internal gears of the drive motor to remain engaged. For example, the rotational movement of the drive motor creates oscillatory translational movement of the top portion. The top portion of the rockable bed frame may apply a force to the drive motor when the top portion changes translational directions at the end of the range of motion. In a conventional oscillatory system without an anti-backlash device, the force applied by the inertia of the top portion can result in a backlash and abrupt movement. Abrupt movement of the top portion may be unpleasant for a user.

In other embodiments, the inertia of the top portion relative to the drive motor and/or base portion may be controlled and/or limited by a biasing element positioned in contact with the top portion and the base portion. For example, an embodiment of a rockable bed frame in accordance with the present disclosure may include a biasing element positioned in contact with the base portion and the top portion and applying a compressive force compressing the linear drive motor between the base portion and the top portion. While a linear drive motor is described, a rotary drive motor or other motive force may be used.

As the top portion translates in the oscillatory movement indicated by the arrow, the top portion changes direction at the ends of the range of movement. Particularly, at the far side relative to the linear drive motor, any movement or tolerance in the connection of the top portion to the linear drive motor may result in a backlash that is unpleasant to a user. The biasing element, such as a spring, a compressible element, a piston-and-cylinder, a magnet, or other device that applies a compressive force in response to extension, may apply a compressive force that urges the top portion toward the linear drive motor, mitigating and/or preventing backlash.

In yet another example of a rockable bed frame with an anti-backlash device in accordance with the present disclosure, a rockable bed frame includes a base portion and a top portion. The top portion translates relative to the base portion upon an application of force from at least a first drive motor and a second drive motor. In some embodiments, the first drive motor is a rotary drive motor, and in other embodiments, the first drive motor is a linear drive motor. The second drive motor may be a rotary drive motor, or the second drive motor may be a linear drive motor. The first drive motor and second drive motor may oppose one another, such that the first drive motor and second drive motor alternately act as the motive force on the top portion and the anti-backlash device on the opposing drive motor.

For example, the first drive motor may apply an expansive force to move the top portion toward the right, while the second drive motor may retard the motion of the top portion and maintain the gears of the first drive motor in engagement and any movement within the connection of the first drive motor and the base portion and/or top portion to a minimum to reduce and/or prevent backlash. In other examples, the first drive motor may apply a contraction force to move the top portion toward the left, while the second drive motor may retard the motion of the top portion and maintain the gears of the first drive motor in engagement and any movement within the connection of the first drive motor and the base portion and/or top portion to a minimum to reduce and/or prevent backlash.

In other embodiments, a rockable bed frame may have a base portion and a top portion movable relative to the base portion by a plurality of electrodynamic suspension coils. The electrodynamic suspension coils may be selectively activated to generate a magnetic field. The direction and magnitude of the magnetic field is controllable by altering the electric current and/or voltage applied to the electrodynamic suspension coils. The magnetic field may generate a magnetic force on a complementary magnetic mount. For example, the electrodynamic suspension coils and the magnetic mounts may be positioned in each corner of the rockable bed frame to support the top portion above the base portion. The magnetic mounts may include or be a magnetic or ferromagnetic material, such that exposure to a magnetic field from the electrodynamic suspension coils may create a magnetic force between the magnetic mount and the electrodynamic suspension coils. In some embodiments, the magnetic mounts may further include or be a flexible or resilient member, such as a spring, a compressible gas, a bushing, a tether or other object positioned between the base portion and the top portion. In other embodiments, the magnetic mounts may further include rails, tracks, or other guidance elements, such as described in relation to embodiments illustrated herein, to allow movement of the top portion relative to the bottom portion in predetermined paths in response to a magnetic force.

In some embodiments, the electrodynamic suspension coils may include electrodynamic suspension coils positioned such that the selective activation of one or more of the electrodynamic suspension coils may apply a magnetic force in one or more axes relative to the top portion of the rockable bed frame. A rockable bed frame may have three electrodynamic suspension coils positioned in each corner of the top portion and a magnetic mount connected to the base portion. In other examples, a rockable bed frame may have three electrodynamic suspension coils positioned in each corner of the base portion and a magnetic mount connected to the top portion. A first electrodynamic suspension coil may be positioned above the magnetic mount in the z-direction. When activated, the first electrodynamic suspension coil may create a magnetic field that interacts with the magnetic mount to apply a z-direction magnetic force in the direction of the first arrow. The z-direction magnetic force may be used to tilt the top portion relative to the base portion.

A second electrodynamic suspension coil may be positioned in the x-direction relative to the first electrodynamic suspension coil, such that the second electrodynamic suspension coil is positioned above the magnetic mount in the z-direction and displaced in the x-direction. When activated, the second electrodynamic suspension coil may create a magnetic field that interacts with the magnetic mount to apply at least a x-direction magnetic force in the direction of the second arrow. The x-direction magnetic force may be used to translate the top portion laterally (in the side-to-side direction from the perspective of a user) relative to the base portion.

A third electrodynamic suspension coil may be positioned in the y-direction relative to the first electrodynamic suspension coil, such that the third electrodynamic suspension coil is positioned above the magnetic mount in the z-direction and displaced in the y-direction. When activated, the third electrodynamic suspension coil may create a magnetic field that interacts with the magnetic mount to apply at least a y-direction magnetic force in the direction of the third arrow. The y-direction magnetic force may be used to translate the top portion longitudinally (in the head-to-toe direction from the perspective of a user) relative to the base portion.

What is claimed is:

1. A bed frame system, comprising:
a base portion;
a top portion disposed over the base portion and moveably connected thereto; and
a linear drive unit fixedly attached to the base portion and to the top portion, wherein the linear drive unit causes one or more of a translational movement, a rotational movement, and an elevational movement of the top portion with respect to the base portion to produce a kinetic input.

2. The system of claim 1, further comprising a computing device in data communication with the linear drive unit.

3. The system of claim 1, one or more of an amplitude, a velocity curve, and a frequency of the translational movement, the rotational movement, and the elevational movement of the top portion with respect to the base portion being adjustable over a time period.

4. The system of claim 1, one or more of an amplitude, a velocity curve, and a frequency of the translational movement, the rotational movement, and the elevational movement of the top portion with respect to the base portion being adjustable independently of the another of the amplitude, velocity curve, and frequency of the top portion with respect to the base portion.

5. The system of claim 1, the linear drive unit including a shaft moved in a longitudinal direction of the linear drive unit by a worm gear.

6. The system of claim 1, the linear drive unit including a shaft moved in a longitudinal direction of the linear drive unit by a piston and cylinder.

7. The system of claim 1, the linear drive unit including a shaft moved in a longitudinal direction of the linear drive unit by a magnetic linear actuator.

8. A method for rocking a bed, the method comprising:
applying a force between a top portion and a base portion of a bed with a linear drive unit;
moving the top portion relative to the base portion with an amplitude, velocity curve, and frequency; and
adjusting one or more of the amplitude, velocity curve, and frequency independently of another of the amplitude, velocity curve, and frequency.

9. The method of claim 8, the amplitude being adjusted independently of the frequency.

10. The method of claim 8, the velocity curve being adjusted independently of the frequency.

11. The method of claim 8, the velocity curve being adjusted independently of the amplitude.

12. The method of claim 8, moving the top portion including moving the top portion with a first amplitude in a first direction relative to the base portion and with a second amplitude in a second direction relative to the base portion, the first amplitude being different from the second amplitude and the second direction being opposite the first direction.

13. The method of claim 8, further comprising receiving information about a user from one or more sensors or monitors.

14. The method of claim 13, adjusting one or more of the amplitude, velocity curve, and frequency including adjusting one or more of the amplitude, velocity curve, and frequency based on the information about the user.

15. The method of claim 8, adjusting one or more of the amplitude, velocity curve, and frequency including adjusting one or more of the amplitude, velocity curve, and frequency over time based on a user selected movement recipe.

16. A system for rocking a user, the system comprising:
a rockable bed frame, the rockable bed frame including:
a base portion,
a top portion disposed over the base portion and moveably connected thereto, and
a linear drive unit fixedly attached to the base portion and to the top portion, wherein the linear drive unit causes one or more of a translational movement, a rotational movement, and an elevational movement of the top portion with respect to the base portion to produce a kinetic input;
a computing device in data communication with the linear drive unit, the computing device including;
processing resources, and
memory resources; and
one or more sensors or monitors in data communication with the computing device.

17. The system of claim 16, the one or more sensors or monitors including a heart rate monitor.

18. The system of claim 16, the memory resources having stored thereon instructions that, when executed by the processing resources, cause the processor to:
apply a force with the linear drive unit between the top portion and the base portion,
move the top portion relative to the base portion, and
adjust one or more of amplitude, velocity curve, and frequency independently of another of the amplitude, velocity curve, and frequency.

19. The system of claim 18, the instructions further including adjusting one or more of the amplitude, velocity curve, and frequency independently of another of the amplitude, velocity curve, and frequency based on information received from the one or more sensors or monitors.

20. The bed frame of claim 1, further comprising:
a processor and memory;
one or more sensors, the one or more sensors including a heart rate monitor;
wherein the base portion includes:
a first track fixedly attached to the base portion and extending at least partially across a width thereof; and
a second track fixedly attached to the base portion and extending at least partially across the width thereof, wherein the first track is oriented parallel to the second track and the first and second tracks define an axis of movement;
wherein an angle of at least a head area or a foot area of the top portion of the bed frame is adjustable by the user, and wherein the top portion includes:
at least one roller disposed in the first track to provide for movement of the top portion with respect to the base portion along the axis of movement defined by the first and second tracks;
at least a second roller disposed in the second track to provide for movement of the top portion with respect to the base portion along the axis of movement defined by the first and second tracks; and
a yoke extending at least partially across a width of the top portion;
wherein the linear drive is attached to the top portion at the yoke, and wherein the translational movement is along the axis of movement defined by the first and second tracks; and
wherein the memory includes programmed instructions to cause the processor to:
determine a natural heart rate of a user;
determine a target heart rate of the user; and
cause the bed frame to provide the kinetic input to the user to adjust the natural heart rate to the target heart rate, wherein the kinetic input is changed incrementally over a time period, and wherein the time period ends when the natural heart rate reaches the target heart rate; and
cause the bed frame to cease providing kinetic input while the natural heart rate is within a predetermined amount of the target heart rate.
